(19) United States
(12) Patent Application Publication
(10) Pub. No.: US 2019/0375707 A1
(43) Pub. Date: Dec. 12, 2019

(54) PROCESS FOR THE PREPARATION OF CHIRAL PYROLLIDINE-2-YL- METHANOL DERIVATIVES

(71) Applicant: Hoffmann-La Roche Inc., Little Falls, NJ (US)

(72) Inventors: Raffael Josef Koller, Basel (CH); Christophe Pfleger, Basel (CH)

(73) Assignee: Hoffmann-La Roche Inc., Little Falls, NJ (US)

(21) Appl. No.: 16/547,134

(22) Filed: Aug. 21, 2019

Related U.S. Application Data
(63) Continuation of application No. PCT/EP2018/054056, filed on Feb. 20, 2018.

(30) Foreign Application Priority Data
Feb. 21, 2017 (EP) 17157035.1

(51) Int. Cl. C07D 207/08

(52) U.S. Cl. CPC C07D 207/08

(57) ABSTRACT

The invention relates to a novel process for the preparation of a chiral pyrollidine-2-yl-methanol derivative or a salt thereof of the formula I

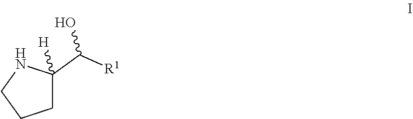

wherein R¹ is aryl or heteroaryl and both aryl or heteroaryl are optionally substituted by C₁₋₄-alkyl, halo-C₁₋₄-alkyl, C₁₋₄-alkoxy or halogen. The chiral pyrollidine-2-yl-methanol derivatives of the formula I are versatile building blocks in the synthesis of pharmacologically active compounds, such as for the stereospecific synthesis of oligonucleotides carrying chiral phosphonate moieties (see e.g. Int. PCT Publication WO 2010/064146).

### CROSS-REFERENCE TO RELATED APPLICATIONS

This application is a continuation of International Application No. PCT/EP2018/054056, filed Feb. 20, 2018, claiming priority to European Application No. 17157035.1, filed Feb. 21, 2017, the contents of each of which are incorporated herein by reference in their entirety.

### SUMMARY

The invention relates to a novel process for the preparation of a chiral pyrollidine-2-yl-methanol derivative or a salt thereof of the formula I

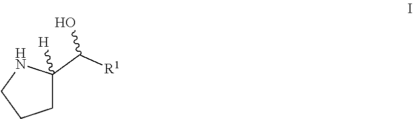

wherein R¹ is aryl or heteroaryl and both aryl or heteroaryl are optionally substituted by C₁₋₄-alkyl, halo-C₁₋₄-alkyl, C₁₋₄-alkoxy or halogen.

Chiral pyrollidine-2-yl-methanol derivatives of the formula I are versatile building blocks in the synthesis of pharmacologically active compounds, such as for the stereospecific synthesis of oligonucleotides carrying chiral phosphonate moieties (see e.g. Int. PCT Publication WO 2010/064146).

A process for the preparation of chiral pyrollidine-2-yl-methanol derivatives of the formula I has been described in Soai et al.; J. Chem. Soc., Chem. Commun. 1986, 412-413. The three step process starts from S-proline and is characterized by the reduction of a chiral benzoyl pyrrolidine with various reducing agents which affords, depending on the reducing agent, erythro/threo mixtures of the chiral pyrollidine-2-yl-methanol.

There is a need for a scalable process which affords the desired chiral building block in good yields and high enantiomeric purity. Object of the invention accordingly was to overcome the flaws of the state of the art processes.

The object is achieved with the new process as described below.

### DETAILED DESCRIPTION

The novel process for the preparation of a chiral pyrollidine-2-yl-methanol derivative or a salt thereof of the formula I

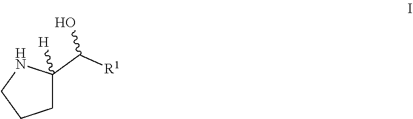

wherein R¹ is aryl or heteroaryl and both aryl or heteroaryl are optionally substituted by C₁₋₄-alkyl, halo-C₁₋₄-alkyl, C₁₋₄-alkoxy or halogen;

comprises the steps

a) a pyrrolidine carboxylic acid derivative of formula II

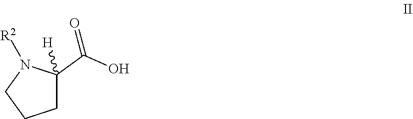

wherein R² is an amino protecting group is transformed with an N,O-dialkylhydroxylamine of the formula V

R⁴ONHR³ V

wherein R³ and R⁴ independently of each other are C₁₋₄-alkyl into the carbamoyl pyrrolidine derivative of formula III

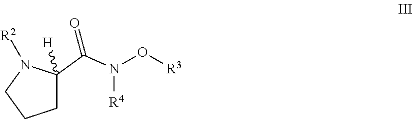

wherein R² is as above and R³ and R⁴ independently of each other are C₁₋₄-alkyl;

b) the carbamoyl pyrrolidine derivative of formula III is reacted with a Grignard reagent of the formula

R¹MgHal

wherein R¹ is as above and Hal stands for a halogen atom to form the aroyl pyrrolidine derivative of formula IV

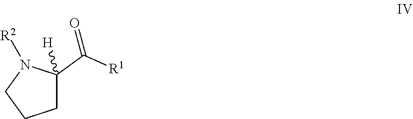

wherein R¹ and R² are as above and;

c) the aroyl pyrrolidine derivative of formula IV is first freed from the amino protecting group R² and subsequently hydrogenated in the presence of a hydrogenation catalyst to form the chiral pyrollidine-2-yl-methanol derivative of the formula I.

The following definitions are set forth to illustrate and define the meaning and scope of the various terms used to describe the invention herein.

The term “chiral” signifies that the molecule can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates.

In a preferred embodiment of the invention the term “chiral” denotes optically pure enantiomers.

In the structural formulae presented herein a broken bond (a) denotes that the substituent is below the plane of the paper and a wedged bond (b) denotes that the substituent is above the plane of the paper.

a) [symbol] b)

[symbol]

The spiral bond (c) denotes both options i.e. either a broken bond (a) or a wedged bond (b).

c)

[symbol]

The term “aryl” denotes a monovalent aromatic carbocyclic mono- or bicyclic ring system comprising 6 to 10 carbon ring atoms. Examples of aryl moieties include phenyl and naphthyl. Phenyl is the preferred aryl group.

The term “heteroaryl” denotes a monovalent aromatic heterocyclic mono or bicyclic ring system of 5 to 12 ring atoms, comprising 1, 2, 3 or 4 heteroatoms selected from N, O and S, the remaining ring atoms being carbon. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl.

Preferably the term “heteroaryl” denotes a monovalent aromatic heterocyclic monocyclic ring system of 5 to 6 ring atoms comprising 1 to 3 heteroatoms selected from N, O and S, the remaining ring atoms being carbon. Examples of preferred heteroaryl moieties include pyrrolyl, furanyl, thienyl, imidazolyl, oxazolyl, thiazolyl, pyridinyl, pyrazinyl, pyrazolyl, pyridazinyl, pyrimidinyl or isoxazolyl.

The term “optionally substituted” in connection with the term “aryl” or “heteroaryl” denotes that the aryl or heteroaryl group may be unsubstituted or substituted by one or more substituents, independently selected from C₁₋₄-alkyl, halo-C₁₋₄-alkyl, C₁₋₄-alkoxy or halogen, preferably from C₁₋₄-alkyl, halo-C₁₋₄-alkyl or C₁₋₄-alkoxy.

The term “C₁₋₄-alkyl” denotes a monovalent linear or branched saturated hydrocarbon group of 1 to 4 carbon atoms. Examples of C₁₋₄-alkyl include methyl, ethyl, propyl, isopropyl, n-butyl, iso-butyl, sec-butyl, or tert-butyl.

The term “C₁₋₄-alkoxy” denotes a group of the formula —O—R′, wherein R′ is a C₁₋₄-alkyl group. Examples of C₁₋₄-alkoxy moieties include methoxy, ethoxy, isopropoxy, and tert-butoxy.

The term “halo”, “halogen”, and “halide” are used interchangeably herein and denote fluoro, chloro, bromo, or iodo.

The term “halo-C₁₋₄-alkyl” denotes a C₁₋₄-alkyl group as defined above which carries one or more halogen substituents as defined above. Examples of halo-C₁₋₄-alkyl are chloromethyl, 2-chloroethyl, 3-chloropropyl, bromomethyl, 2-bromoethyl, 3-bromopropyl, 2,2-dichloroethyl, trichloromethyl or trichloroethyl.

In a preferred embodiment of the present invention R¹ is aryl, preferably phenyl or naphthyl, more preferably phenyl unsubstituted or substituted by C₁₋₄-alkyl, halo-C₁₋₄-alkyl, C₁₋₄-alkoxy or halogen.

In another preferred embodiment R¹ is phenyl unsubstituted or substituted by one or more substituents selected from methyl, ethyl, propyl, isopropyl, n-butyl, iso-butyl, sec-butyl, tert-butyl, fluoro, chloro, bromo, iodo, methoxy, ethoxy, isopropoxy or tert-butoxy.

Preferred examples for R¹ are phenyl, naphthyl, p-tolyl, m-tolyl, 3,5-difluorophenyl, 3,4,5-trifluorophenyl or 3,5-dimethoxyphenyl.

Phenyl is the most preferred substituent for R¹.

The term “amino-protecting group” denotes groups intended to protect an amino group and includes benzyl, benzyloxycarbonyl (carbobenzyloxy, CBZ), Fmoc (9-Fluorenylmethyloxycarbonyl), p-methoxybenzyloxycarbonyl, p-nitrobenzyloxycarbonyl, tert-butoxycarbonyl (BOC), and trifluoroacetyl. Further examples of these groups are found in T. W. Greene and P. G. M. Wuts, “Protective Groups in Organic Synthesis”, 2nd ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981.

Preferred amino-protecting group is tert-butoxycarbonyl (BOC).

The term “salt” in the context of the present invention denotes the salt generated upon removal of the amino-protecting group R², i.e. salts formed with a strong acid such as with hydrochloric acid or trifluoro acetic acid. The hydrochloride salt formed with hydrochloric acid is the preferred salt.

In a preferred embodiment of the present invention the chiral pyrollidine-2-yl-methanol derivative of the formula I has the structure Ia.

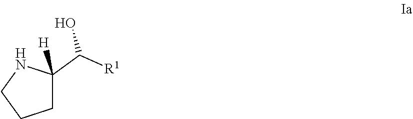

wherein R¹ is as above.

In another preferred embodiment of the present invention the chiral pyrollidine-2-yl-methanol derivative of the formula I has the structure Ib.

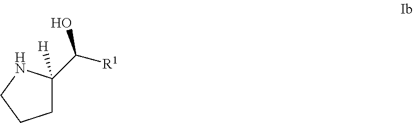

wherein R¹ is as above.

Preferably, the substituent R¹ stands for phenyl, optionally substituted by C₁₋₄-alkyl, C₁₋₄-alkoxy or halogen, but particularly for unsubstituted phenyl.

Step a)

Step a) requires the transformation of a pyrrolidine carboxylic acid derivative of formula II with a N,O-dialkylhydroxylamine into the carbamoyl pyrrolidine derivative of formula III.

The pyrrolidine carboxylic acid derivatives of formula II are particularly used in their chiral form preferably as pure enantiomers.

The amino protecting group R² can be selected from those mentioned above, but preferred are those which are cleavable under strong acidic conditions. Preferred amino protecting group is tert-butoxycarbonyl (BOC).

The N,O-dialkylamine has the formula V

R⁴ONHR³ V

wherein R³ and R⁴ independently of each other are C₁₋₄-alkyl is usually applied in the form of a suitable salt such as the hydrochloride.

In a preferred embodiment R³ and R⁴ are methyl.

The coupling as a rule takes place in the presence of a coupling agent, an amine base and an organic solvent at a reaction temperature between 0° C. and 60° C.

The coupling agent can be selected from DCC (N,N′-dicyclohexylcarbodiimide) or EDC (N-(3-dimethylaminopropyl)-N′-ethylcarbodiimide-hydrochloride) or TBTU (N,N,N′,N′-tetramethyl-O-(benzotriazol-1-yl)uronium tetrafluoroborate, HBTU (2-(1H-benzotriazol-1-yl)-1,1,3,3-tetramethyluronium hexafluorophosphate) together with an additive selected from HOBt (1-hydroxybenztriazole), HOSu (N-hydroxysuccinimide) or HOAt (1-hydroxy-7-azabenzotriazole and common combinations thereof such as TBTU/HOBt or HBTU/HOAt.

A suitable alternative is n-propylphosphonic acid anhydride (T3P®).

The amine base usually is a tertiary amine, like triethylamine or N-ethyldiisopropylamine, pyridine derivatives such as 2,4,6-collidine, DABCO (1,4-Diazabicyclo[2.2.2]octane) or N-methylmorpholine, but preferably N-methylmorpholine.

Preferably n-propylphosphonic acid anhydride (T3P®) is used as coupling agent.

The reaction expediently takes place in a polar aprotic solvent like acetonitrile, dimethyl sulfoxide or tetrahydrofuran or mixtures thereof.

The reaction temperature preferably is in the range of 10° C. and 40° C., more preferably at 20° C. to 30° C.

Isolation of the formed carbamoyl pyrrolidine derivative of formula III can happen by methods known to the skilled in the art such as by adding water and a weak acid and subsequent extraction with a suitable organic solvent like ethylacetate or toluene. Weak acids can be selected from an organic acid like citric acid or from diluted mineral acids like diluted hydrochloric-, sulfuric or phosphoric-acid. Evaporation of the organic solvent after extraction with a weak base as a rule provides the carbamoyl pyrrolidine derivative of formula III in a sufficient purity for the next step. Alternatively, the pyrrolidine derivative of formula III can also be used as solutions in toluene or THF for the next step.

Preferred carbamoyl pyrrolidine derivatives have the formula IIIa or IIIb

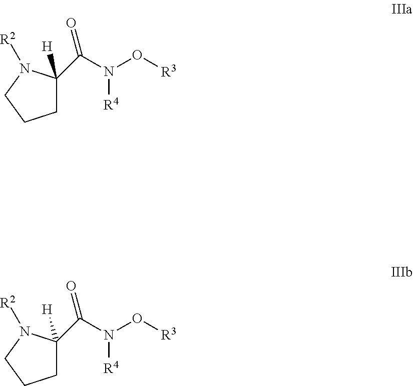

wherein R², R³ and R⁴ are as above.

Even more preferred carbamoyl pyrrolidine derivatives have the formula IIIc or IIId

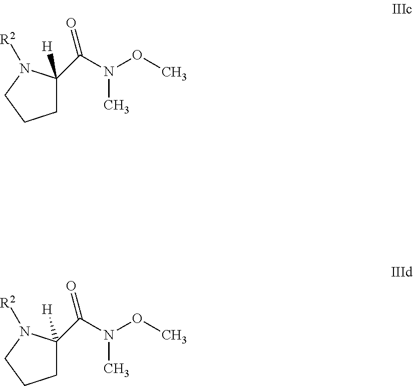

wherein R² is as above.

In a still further preferred embodiment R² is tert-butoxycarbonyl (BOC).

Step b)

Step b) requires the reaction of the carbamoyl pyrrolidine derivative of formula III with a Grignard reagent to form the aroyl pyrrolidine derivative of formula IV.

In view of the fact that initial step a) is preferably performed with a chiral starting compound the starting compound for step b) i.e. the carbamoyl pyrrolidine derivative of formula III is also particularly used in their chiral form preferably as pure enantiomer.

The Grignard reaction can be performed following methods well known to the skilled in the art.

The Grignard reagents of the formula R¹MgHal, wherein R¹ is as above and Hal stands for a halogen atom are either commercially available or can alternatively readily be prepared as described e.g. by P. Knochel and co-workers in Angew. Chem. Int. Ed., 2004, 43, 3333-3336.

In a preferred embodiment R¹ is phenyl optionally substituted by C₁₋₄-alkyl, preferably methyl or ethyl, C₁₋₄-alkoxy, preferably methoxy or ethoxy or halogen, preferably fluorine. More preferably R¹ is unsubstituted phenyl.

Hal preferably stands for chlorine or bromine.

The most preferred Grignard reagent is PhenylMgBr.

The Grignard reaction is usually performed in an organic solvent, preferably an ethereal or aromatic hydrocarbon solvent or mixtures thereof. Typical ethereal solvent are tetrahydrofuran, methyl-tetrahydrofuran or cyclopentyl methyl ether. A typical aromatic solvent is toluene.

The reaction temperature is commonly selected between −10° C. and 50° C., but typically lower temperatures between 0° C. and 30° C. are preferred.

Isolation of the aroyl pyrrolidine derivative of formula IV can happen following methods known to the skilled in the art, for instance by quenching the reaction mixture with a weak acid such as with an aqueous organic acid like citric acid or an aqueous mineral acid. Subsequent extraction of the biphasic mixture with a suitable organic solvent which can be selected from hydrocarbons like heptane, ethers like tetrahydrofuran or aromatic solvents like toluene and finally evaporation of the organic phase renders the crude aroyl pyrrolidine. Further purification can be reached by crystallization in a polar protic solvent like an aqueous i-propanol or n-propanol.

In view of the preference to use chiral compounds the preferred aroyl pyrrolidine derivative have the formula IVa or IVb.

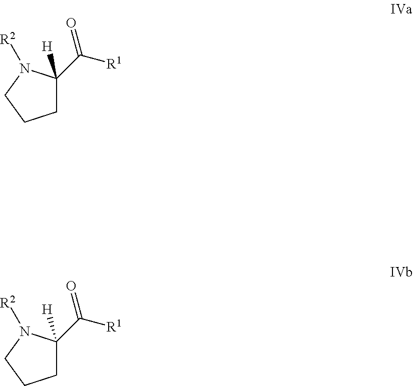

wherein R¹ and R² are as above.

Step c)

Step c) requires in a first step the removal of the amino protecting group R² in the aroyl pyrrolidine derivative of the formula IV and in a second step the hydrogenation in the presence of a hydrogenation catalyst to form the chiral pyrollidine-2-yl-methanol derivative of the formula I.

The removal of amino protecting groups can be accomplished following methods known in literature and to the skilled in the art. The preferred amino protecting groups are those which are cleavable with a strong acid.

Suitable strong acids are mineral acids such as hydrochloric acid or a strong organic acid such as trifluoroacetic acid, however typically an aqueous hydrochloric acid having a HCl concentration of 25% and more is used.

The reaction usually takes place in the presence of a protic solvent, for instance in lower alcohols like ethanol or n-propanol at elevated temperatures between 40° C. to 80° C. until no starting material can any longer be detected.

The reaction mixture can then, without isolation of the de-protected intermediate (which in case of HCl is the hydrochloride salt of the de-protected aroyl pyrrolidine) be transferred to the hydrogenation reaction.

The hydrogenation is performed in the presence of a hydrogenation catalyst, preferably consisting of a platinum group metal selected from ruthenium, osmium, rhodium, iridium, palladium and platinum preferably from palladium.

The platinum metals are usually applied on an inert carrier, typically on carbon.

In a preferred embodiment palladium (Pd) on carbon, more preferably 2% wt. Pd to 20% wt. Pd, even more preferably 8% wt. to 12% wt. Pd on carbon is used.

The hydrogenation reaction expediently takes place in a polar protic solvent at a reaction temperature between 0° C. and 60° C. and a hydrogen pressure between 1 bar and 10 bar.

The polar protic solvent is preferably the same as used for the de-protection, i.e. a lower alcohol like ethanol or n-propanol.

The reaction temperature preferably is maintained between 20° C. and 40° C. and the hydrogen pressure preferably is selected between 3 bar and 7 bar.

After completion of the reaction the catalyst is removed by filtration. The desired product can then be obtained by crystallization with a suitable solvent such as with n-propylacetate or i-propylacetate.

Further purification can be reached by a recrystallization from a polar aprotic solvent, preferably from acetonitrile.

According to the preferred embodiment the chiral aroyl pyrrolidines of formula IVa or IVb are applied for step c). The desired pyrollidine-2-yl-methanol derivative of the formula I, particularly of formula Ia or Ib can following the methods described above be obtained in high yields and an optical purity of greater 95% ee, preferably greater 99% ee.

The desired pyrollidine-2-yl-methanol derivative of the formula I, particularly of formula Ia or Ib, are obtained in the form of the salt of the strong acid used for the de-protection of the preferred BOC-group as described above, preferably the hydrochloride salt.

### EXAMPLES

### Abbreviations

rt=room temperature, T3P®=propylphosphonic anhydride, EtOAc=ethyl acetate, NMM=4-methylmorpholine, ACN=acetonitrile, PhMgBr=phenylmagnesium bromide, THF=tetrahydrofuran, CPME=cyclopentyl methyl ether, n-PrOH=1-Propanol, i-PrOH=2-propanol, n-PrOAc=propyl acetate, TFA=trifluoroacetic acid

### Example 1

### Preparation of (R)-phenyl-[(2S)-pyrrolidin-2-yl]methanol Hydrochloride

Reaction Scheme:

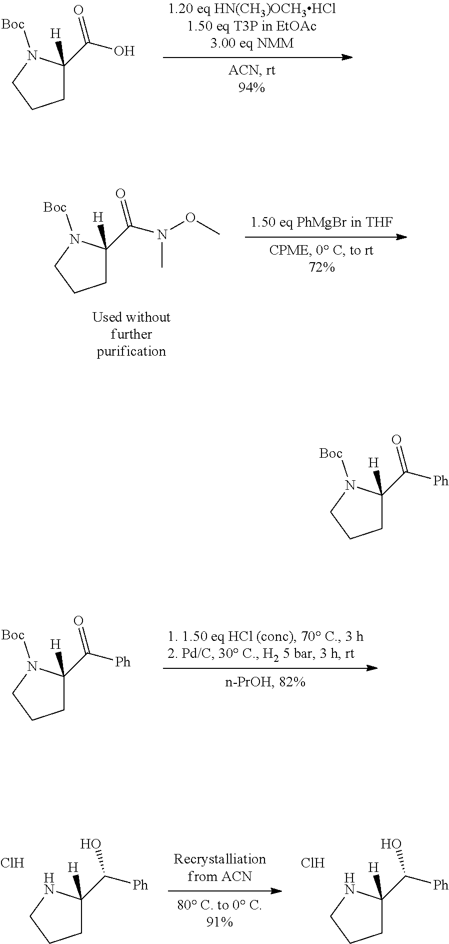

### a) tert-Butyl (2S)-2-[methoxy(methyl)carbamoyl]pyrrolidine-1-carboxylate

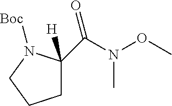 [SMILES: CON(C)C(=O)[C@@H]1CCCN1C(=O)OC(C)(C)C]

A 500-mL-round-bottomed flask equipped with an overhead stirrer was charged with (S)-1-(tert-butoxycarbonyl)pyrrolidine-2-carboxylic acid (50 g, 232 mmol, Eq: 1) and N,O-dimethyl-hydroxylamine hydrochloride (27.2 g, 279 mmol, Eq: 1.2). Under an inert atmosphere the solids were suspended in acetonitrile (354 g, 450 ml, Eq: -) to give a light yellow suspension. N-methylmorpholine (70.5 g, 76.6 ml, 697 mmol, Eq: 3) was added dropwise over 40 min at rt. During the addition the reaction was kept a rt. To the resulting suspension 1-propanephosphonic anhydride in EtOAc (50%, 222 g, 205 ml, 348 mmol, Eq: 1.5) was added over 40 min keeping the reaction mixture at rt. After the addition the suspension was stirred for 2 h at rt, diluted with water (175 mL) and stirred for 30 min before citric acid (325 ml 1.6 M, 520 mmol) was added. The resulting clear yellow solution was extracted three times with EtOAc (500 mL each). The organic phases were washed twice with 5% NaHCO₃ (625 mL each), followed by 10% NaCl solution (625 mL). The combined organic phases were concentrated under reduced pressure and the oily residue was suspended in toluene (500 mL), filtered and the clear solution was again concentrated under reduced pressure to give 56.3 g (94%) of tert-butyl (2S)-2-[methoxy(methyl)carbamoyl]-pyrrolidine-1-carboxylate as a clear yellowish oil with a chemical purity of 95.0% (see GC method bellow) and enantiomeric excess >99.9% (see chiral HPLC method below).

1H-NMR (600 MHz, CDCl₃) δ ppm 4.59 (br s, 1H), 3.67-3.82 (m, 3H), 3.53-3.64 (m, 1H), 3.36-3.51 (m, 1H), 3.11-3.24; (m, 3H), 2.07-2.27 (m, 1H), 1.77-2.07 (m, 3H), 1.34-1.49 (m, 9H).

GC method: Column: HP-5, 30 m×0.32 mm ID, 0.25 μm; Temp: 50° C. to 150° C., 10° C./min, 150° C. to 250° C., 20° C./min, at 250° C. hold up for 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL ACN. Retention time: 12.41 min tert-butyl (2S)-2-[methoxy(methyl)-carbamoyl]pyrrolidine-1-carboxylate.

Chiral HPLC method: Column: Chiralpak IC-3, 150×4.6 mm, 3 um, Nr. 188; Mobile phases, A: n-heptane, 80%, B: 0.1% TFA in n-heptane, 10%, C: Ethanol, 10%; Flow: 2.5 mL/min isocratic; Temp.: 40° C.; Starting Pressure: 186 bar; Inj. Vol.: 4.0 uL; UV 210 nm; Sample prep: 5 mg/ml Ethanol. Retention times: 2.82 min tert-butyl (2S)-2-[methoxy(methyl)carbamoyl]-pyrrolidine-1-carboxylate, 3.26 min tert-butyl (2R)-2-[methoxy(methyl)carbamoyl]pyrrolidine-1-carboxylate.

### b) tert-Butyl (2S)-2-benzoylpyrrolidine-1-carboxylate

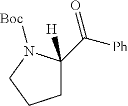 [SMILES: CC(C)(C)OC(=O)N1CCC[C@H]1C(=O)c1ccccc1]

A 500-mL-round-bottomed flask equipped with an overhead stirrer was charged with (S)-tert-butyl 2-(methoxy(methyl)carbamoyl)pyrrolidine-1-carboxylate (26.18 g, 99.9 mmol, Eq: 1) in cyclopentyl methyl ether (100 mL). The clear solution was cooled to 0° C. and phenylmagnesium bromide (1.0M in THF, 150 ml, 150 mmol, Eq: 1.5) was added dropwise over 30 min maintaining the temperature at 0° C. The resulting light brown clear solution was stirred for 80 min at 0° C., then warmed to rt over 1 hr and stirred for 2 h and 20 min at rt. After 25 min at rt the clear solution became turbid.

The reaction mixture was cooled to 0° C. and carefully quenched with citric acid (200 mL, 1.6M, 230 mmol). The resulting biphasic mixture was allowed to separate and the organic, yellow clear solution was separated and the aqueous layer was extracted with heptane (100 mL). The organic layers were washed twice with 5% NaHCO₃ (250 mL each) and 10% NaCl (200 mL), combined, dried over Na₂SO₄, filtered and evaporated under reduced pressure to give 24.6 g of a clear, dark yellow oil with a chemical purity of 79.8% (see GC method below).

The crude material was dissolved in a mixture of i-PrOH (100 mL) and water (100 mL) at 50° C. to give a yellow clear solution. The solution was cooled to 0° C. over 3 h, seeded at 40° C., 35° C., and 30° C. with 200 mg pure material. To the suspension at 0° C. water (85 mL) was added over 1 hr. After complete addition the yellow suspension was stirred for 1 hr at 0° C. The crystals (light yellow) were filtered, washed with a mixture of i-PrOH/water (3.5:6.5, 100 mL) and dried under reduced pressure. After drying for 14 h 19.7 g (71.6%) off-white crystals were obtained with a chemical purity of 99.4% (see GC method below) and an enantiomeric excess of >99.9% (see chiral SFC method below).

1H NMR (600 MHz, CHLOROFORM-d) δ ppm 7.89-8.11 (m, 2H), 7.42-7.67 (m, 3H), 5.12-5.47 (m, 1H), 3.39-3.79 (m, 2H), 2.21-2.49 (m, 1H), 1.81-2.03 (m, 3H), 1.46 (s, 9H);

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H2); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL ACN. Retention times: 12.41 min tert-butyl (2S)-2-[methoxy(methyl)-carbamoyl]pyrrolidine-1-carboxylate, 14.09 min tert-butyl (2S)-2-benzoylpyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.47 min tert-Butyl (2R)-2-benzoylpyrrolidine-1-carboxylate, 1.66 min tert-butyl (2S)-2-[methoxy(methyl)carbamoyl]pyrrolidine-1-carboxylate.

### c) (R)-phenyl-[(2S)-pyrrolidin-2-yl]methanol Hydrochloride

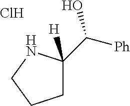 [SMILES: Cl ; O[C@H](c1ccccc1)[C@@H]1CCCN1]

A 350 mL-round-bottomed flask equipped with an overhead stirrer was charged with (S)-tert-butyl 2-benzoylpyrrolidine-1-carboxylate (28.5 g, 104 mmol, Eq: 1) in 1-propanol (114 g, 143 ml, Eq: -). The clear yellow solution was heated to 70° C. and then HCl 37% (15.3 g, 12.7 ml, 155 mmol, Eq: 1.5) was added dropwise over 15 min. The resulting dark yellow clear solution was stirred at 70° C. for 3 hr at which point complete disappearance of the starting material was observed. The reaction mixture was cooled to rt and transferred to an autoclave, the flask was rinsed with additional 1-propanol (11 g, 14 ml, Eq: -) and this solution was also transferred to the autoclave. After establishing an atmosphere of argon Palladium on Carbon (10%, 1.81 g, 1.7 mmol, Eq: 0.02) was added. The autoclave was flushed with H₂ three times, heated to 30° C. and under stirring the hydrogen pressure was increased to 5 bar. After 3 h the reaction mixture was cooled to rt and the autoclave was ventilated. The reaction mixture was filtered and the filter cake washed with 1-propanol (200 ml, Eq: -) The crude reaction mixture showed a mixture of desired (R)-phenyl-[(2S)-pyrrolidin-2-yl]methanol hydrochloride (94.8%), (S)-phenyl-[(2S)-pyrrolidin-2-yl]methanol hydrochloride (3.1%), (S)-phenyl-[(2R)-pyrrolidin-2-yl]methanol hydrochloride (1.0%), (2S)-2-benzylpyrrolidine (see chiral SFC method below).

The reaction mixture was concentrated under reduced pressure to 135 g at which point n-PrOAc (100 mL) was added. The resulting mixture was again concentrated under reduced pressure to 130 g at which point n-PrOAc (100 mL) was added. The resulting suspension was stirred for 2 h at rt, then cooled to 0° C. and stirred for 2 h. The suspension was filtered, and the crystalline white solid was washed with cold (0° C.) n-PrOAc (100 mL). After drying under reduced pressure at 50° C. for 14 h white crystals (18.1 g, 82%) with a chemical purity of 99.1% and an enantiomeric excess of >99% were obtained.

1H NMR (600 MHz, DMSO-d6) δ ppm 8.74-9.85 (m, 1H), 7.48-7.49 (m, 1H), 7.21-7.46 (m, 2H), 6.09 (br d, J=3.5 Hz, 1H), 5.06 (br s, 1H), 3.61-3.79 (m, 1H), 3.07-3.20 (m, 1H), 1.68-1.97 (m, 1H), 1.52-1.65 (m, 1H). HRMS: (ESI-TOF) calculated for (C₁₁H₁₅NO): 177.1154, found: 177.1154.

Chiral SFC method: Chiralcel OZ-3, 150×4.6 mm, Nr: 183; Mobile phases, A: CO₂, 90-60% in 8.8 min, hold for 0.5 min, B: Ethanol+0.2% iso-propyl amine, 10-40% in 8.8 min, hold for 0.5 min; Flow: 3 ml/min; Temp: 50° C.; BPR: 130 bar; Inj. Vol.: 5.0 uL; UV 210 nm; Sample prep.: 2.0 mg/ml Ethanol. Retention times: 2.67 min (2R)-2-benzylpyrrolidine, 3.01 min (2S)-2-benzylpyrrolidine, 3.84 min (S)-phenyl-[(2R)-pyrrolidin-2-yl]methanol, 4.09 min phenyl-[(2R)-pyrrolidin-2-yl]methanone, 4.33 min phenyl-[(2S)-pyrrolidin-2-yl]methanone, 4.55 min (R)-phenyl-[(2S)-pyrrolidin-2-yl]methanol, 4.89 min (S)-phenyl-[(2S)-pyrrolidin-2-yl]methanol, 5.47 min (R)-phenyl-[(2R)-pyrrolidin-2-yl]methanol.

### Purification of (R)-phenyl-[(2S)-pyrrolidin-2-yl]methanol Hydrochloride

A 30 L Reactor was charged with (S)-phenyl((R)-pyrrolidin-2-yl)methanol (1.16 kg, 5.44 mol) and acetonitrile (13.8 kg, 17.5 l, Eq: -). The resulting suspension was heated to 80° C. until a solution was obtained and the volume was reduced by 1 L via destillation. The clear yellow solution was cooled to 0° C. over 3 hr. The resulting suspension was stirred for an additional 1 hr at 0° C. and was then filtered. The white crystals where washed with cold (0° C.) acetonitrile (3.93 kg, 5 l, Eq: -) and dried under reduced pressure to give desired product (1057 g, 91%) with a chemical purity of >99% (see HPLC method bellow) and an enantiomeric excess of >99% (see chiral SFC method bellow)

HPLC method: Column: XBridge BEH Phenyl, 2.5 um, 100×4.6 mm, Nr: 207; Solvent: A: H2O/ACN, 95/5: 80-45% in 4 min, hold for 1 min, B: ACN: 15-50% in 4 min, hold for 1 min, D: 100 mM Ammonium formate in H2O/ACN (95/5) @ pH9 with NH3: 5% isocratic; Temp: 50° C.; Flow: 1.5 ml/min; Inj. Vol.: 3.5 ul+wash, Starting pressure: 247 bar; Detector: 212 nm, BW: 8 nm, Ref: 360 nm, Ref BW: 50M; Sample Prep: 0.5 mg/ml in H2O/ACN (1/1; v/v)

Chiral SFC method: Chiralcel OZ-3, 150×4.6 mm, Nr: 183; Mobile phases, A: CO₂, 90-60% in 8.8 min, hold for 0.5 min, B: Ethanol+0.2% iso-propyl amine, 10-40% in 8.8 min, hold for 0.5 min; Flow: 3 ml/min; Temp: 50° C.; BPR: 130 bar; Inj. Vol.: 5.0 uL; UV 210 nm; Sample prep.: 2.0 mg/ml Ethanol. Retention times: 2.67 min (2R)-2-benzylpyrrolidine, 3.01 min (2S)-2-benzylpyrrolidine, 3.84 min (S)-phenyl-[(2R)-pyrrolidin-2-yl]methanol, 4.09 min phenyl-[(2R)-pyrrolidin-2-yl]methanone, 4.33 min phenyl-[(2S)-pyrrolidin-2-yl]methanone, 4.55 min (R)-phenyl-[(2S)-pyrrolidin-2-yl]methanol, 4.89 min (S)-phenyl-[(2S)-pyrrolidin-2-yl]methanol, 5.47 min (R)-phenyl-[(2R)-pyrrolidin-2-yl]methanol.

### Example 2

### Preparation of (S)-phenyl-[(2R)-pyrrolidin-2-yl]methanol Hydrochloride

Reaction Scheme:

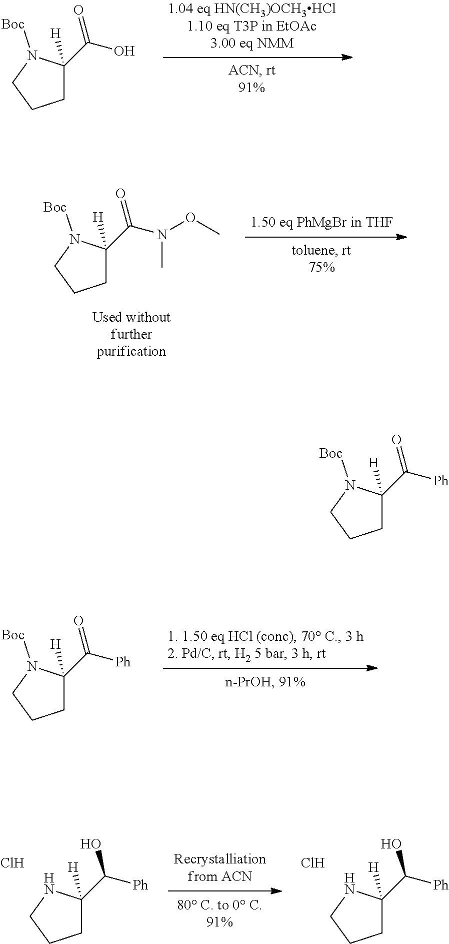

### a) tert-Butyl (2R)-2-[methoxy(methyl)carbamoyl]pyrrolidine-1-carboxylate

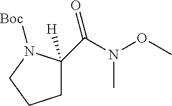 [SMILES: CON(C)C(=O)[C@H]1CCCN1C(=O)OC(C)(C)C]

A 30-L reactor was charged with (R)-1-(tert-butoxycarbonyl)pyrrolidine-2-carboxylic acid (2300 g, 10.7 mol, Eq: 1) and N, O-dimethylhydroxylamine hydrochloride (1.09 kg, 11.1 mol, Eq: 1.04). Under an inert atmosphere the solids were suspended in acetonitrile (16.2 kg, 20.6 l, Eq: -) to give a light yellow suspension. N-methylmorpholine (3.24 kg, 3.52 l, 32.1 mol, Eq: 3) was added dropwise over 15 min at rt. During the addition the reaction was kept a rt. The resulting suspension was stirred for 40 min at rt, before 1-propanephosphonic anhydride in EtOAc (50%, 7.48 kg, 6.93 l, 11.8 mol, Eq: 1.1) was added at rt ° C. over 70 min keeping the reaction mixture at rt. After the addition the suspension was stirred for 2 h at rt and then concentrated at 60° C. under reduced pressure to a total volume of 18 L. The solvent was replaced under reduced pressure with toluene under a constant volume. Toluene (7.71 kg, 9 l, Eq: -) was added to further dilute the suspension before it was filtered and to the resulting clear solution was washed with a solution of citric acid monohydrate (1.84 kg, 8.76 mol, Eq: 0.819) in water (7.36 l, Eq: -), followed by a solution of NaHCO₃ (460 g, 5.48 mol, Eq: 0.512) in water (8.62 L), followed by a solution of NaCl (920 g) in water (8.24 L). Then the organic phase was separated. The aqueous phases were re-extracted with toluene (8.65 kg) and the organic phases were combined, concentrated at 50° C. under reduced pressure to 6 L, filtered and washed with toluene to a give a solution of tert-Butyl (2R)-2-[methoxy(methyl)carbamoyl]pyrrolidine-1-carboxylate (2510 g, 91%) in toluene (3200 g).

To toluene solution was used in the next step without further purification. Analytical data was generated by concentration of an aliquot of the solution under reduced pressure and analyzing the oily residue which yielded a clear yellowish oil with a chemical purity of 98.8% (see HPLC method bellow) and enantiomeric excess >99% (see chiral HPLC method below).

1H NMR (600 MHz, CDCl₃) δ ppm 4.59-4.75 (m, 1H), 3.68-3.85 (m, 3H), 3.35-3.63 (m, 2H), 3.21 (s, 3H), 1.85-2.29 (m, 2H), 1.82-2.06 (m, 2H), 1.38-1.49 (m, 9H).

HPLC method. Column: XBridge BEH C8, 2.5 um, 100×4.6 mm, Nr: 182; Mobile phases: A: H₂O/ACN, 95/5: 80-10% in 6 min, hold for 1 min, B: ACN, 10-80% in 6 min, hold for 1 min, C: H₂O+0.5% TFA: 10% isocratic; Flow: 1.5 ml/min; Temp.: 45° C.; Inj. Volume: 2 ul; UV: 200 nm (BW: 8 nm), Ref: 360 nm (BW: 100 nm); Sample prep: 2 mg/mL H₂O/ACN, 1/1 Chiral HPLC method: Column: Chiralpak IC-3, 150×4.6 mm, 3 um, Nr. 188; Mobile phases, A: n-heptane, 80%, B: 0.1% TFA in n-heptane, 10%, C: Ethanol, 10%; Flow: 2.5 mL/min isocratic; Temp.: 40° C.; Starting Pressure: 186 bar; Inj. Vol.: 4.0 uL; UV 210 nm; Sample prep: 5 mg/ml Ethanol. Retention times: 2.82 min tert-butyl (2S)-2-[methoxy-(methyl)carbamoyl]pyrrolidine-1-carboxylate, 3.26 min tert-butyl (2R)-2-[methoxy-(methyl)carbamoyl]pyrrolidine-1-carboxylate.

### b) tert-Butyl (2R)-2-benzoylpyrrolidine-1-carboxylate

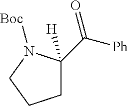 [SMILES: CC(C)(C)OC(=O)N1CCC[C@@H]1C(=O)c1ccccc1]

A 30-L reactor was charged with (R)-tert-butyl 2-(methoxy(methyl)carbamoyl)pyrrolidine-1-carboxylate (2382 g, 9.22 mol, Eq: 1) in toluene (3200 g). Toluene was added (4.35 kg, 5 L), the clear solution was cooled to rt and phenylmagnesium bromide (15% in THF, 17 kg, 17.3 L, 14.1 mol, Eq: 1.53) was added over 60 min maintaining the temperature at rt. The initially clear yellow solution turns brownish over the course of the reaction. After 4 h at rt, the reaction mixture is cooled to 5° C. and added to a solution of citric acid monohydrate (2.1 kg, 10.9 mol, Eq: 1.19) in water (10 L) at 5° C. under stirring over the course of 30 min. The organic phase was separated and washed twice a solution of NaHCO₃ in water (5%, 10 L) followed by a solution of NaCl in water (5%, 10 L). The organic phase was again separated and concentrated under reduced pressure to give a red oil (3.16 kg), which was re-dissolved in toluene (4.33 kg, 5 l, Eq: -), filtered and the resulting solution was diluted with n-propanol (8 kg, 10 l, Eq: -). The solution was filtered over activated charcoal and washed with additional n-propanol (2.4 kg, 3 L, Eq: -) and again concentrated under reduced pressure at 50° C. and the residue redissolved in n-propanol (4.4 kg, 5.5 L, Eq: -). To the clear red solution at 35° C. water (5.5 kg, 5.5 L, Eq: -) was added over 30 min and the resulting solution was seeded with pure tert-Butyl (2R)-2-benzoylpyrrolidine-1-carboxylate. After the crystallisation started additional water (22 kg, 22 L, Eq: -) was added. The resulting suspension was stirred at rt for 30 min and then cooled to 5° C. and stirred for 5 h. The crystalline solid was filtered of and the reactor and the solids were washed with a solution of n-propanol (0.5 L) and water (3.0 L). The solid was dissolved in n-propanol (4 kg, 5 L) and concentrated under reduced pressure at 50° C. to remove residual water, the resulting solid was again dissolved in n-propanol (4 kg, 5 L) and concentrated under reduced pressure at 50° C. to give an orange, crystalline solid (1.90 kg, 75%) with a purify of 99.2% (see GC method below) and enantiomeric excess of >99% (see chiral SFC method)

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H2); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL ACN. Retention times: 12.41 min tert-butyl (2S)-2-[methoxy(methyl)carbamoyl]pyrrolidine-1-carboxylate, 14.09 min tert-butyl (2S)-2-benzoylpyrrolidine-1-carboxylate.

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.47 min tert-Butyl (2R)-2-benzoylpyrrolidine-1-carboxylate, 1.66 min tert-butyl (2S)-2-[methoxy(methyl)carbamoyl]pyrrolidine-1-carboxylate.

### c) (S)-phenyl-[(2R)-pyrrolidin-2-yl]methanol Hydrochloride

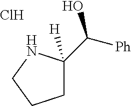 [SMILES: Cl ; O[C@@H](c1ccccc1)[C@H]1CCCN1]

A 4500 mL-round-bottomed flask equipped with an overhead stirrer was charged with (R)-tert-butyl 2-benzoylpyrrolidine-1-carboxylate (476 g, 1.73 mol, Eq: 1) in 1-propanol (1.84 kg, 2.3 l, Eq: -). The clear yellow solution was heated to 60° C. and then HCl 37% (261 g, 221 ml, 2.65 mol, Eq: 1.53) was added dropwise over 15 min. The resulting dark yellow clear solution was stirred at 60° C. for 3 hr and then heated to 70° C. and stirred for another 3 hr at which point complete disappearance of the starting material was observed. The reaction mixture was cooled to rt and transferred to an autoclave, the flask was rinsed with additional 1-propanol (110 g, 140 ml, Eq: -) and this solution was also transferred to the autoclave. After establishing an atmosphere of argon Palladium on Carbon (10%, 18.3 g, 17.2 mmol, Eq: 0.01) was added. The autoclave was flushed with H₂ three times, heated to 25° C. and under stirring the hydrogen pressure was increased to 5 bar. After 3 h the reaction mixture was cooled to rt and the autoclave was ventilated. The reaction mixture was filtered and the filter cake washed with 1-propanol (500 ml, Eq: -). The crude reaction mixture showed the desired (S)-phenyl-[(RS)-pyrrolidin-2-yl]methanol hydrochloride in 97.3% purity (see chiral SFC method below).

The reaction mixture was concentrated under reduced pressure at 60° C. to 1.5 L at which point crystallization of the product already started subsequently the solvent was exchanged with n-PrOAc maintaining a constant volume at 60° C. For the solvent exchange n-PrOAc (8.01 kg, 9 l, Eq: -) was used. The resulting mixture was cooled to rt and stirred for 1 hr at rt. The suspension was filtered, and the crystalline solid was washed with n-PrOAc (623 g, 700 ml, Eq:). After drying under reduced pressure at 50° C. for 14 h yellow crystals (335 g, 91%) with a chemical purity of 98% (see HPLC method below) and an enantiomeric excess of 99% (see chiral SFC method bellow) were obtained.

1H NMR (600 MHz, DMSO-d6) δ ppm 9.40-9.52 (m, 1H), 8.68-8.80 (m, 1H), 7.40-7.43 (m, 2H), 7.36-7.40 (m, 2H), 7.27-7.32 (m, 1H), 6.04-6.12 (m, 1H), 5.01-5.06 (m, 1H), 3.66-3.74 (m, 1H), 3.08-3.19 (m, 2H), 1.54-1.94 (m, 4H)

HPLC method: Column: XBridge BEH Phenyl, 2.5 um, 100×4.6 mm, Nr: 207; Solvent: A: H2O/ACN, 95/5: 80-45% in 4 min, hold for 1 min, B: ACN: 15-50% in 4 min, hold for 1 min, D: 100 mM Ammonium formate in H2O/ACN (95/5) @ pH9 with NH3: 5% isocratic; Temp: 50° C.; Flow: 1.5 ml/min; Inj. Vol.: 3.5 ul+wash, Starting pressure: 247 bar; Detector: 212 nm, BW: 8 nm, Ref: 360 nm, Ref BW: 50M; Sample Prep: 0.5 mg/ml in H2O/ACN (1/1; v/v)

Chiral SFC method: Chiralcel OZ-3, 150×4.6 mm, Nr: 183; Mobile phases, A: CO₂, 90-60% in 8.8 min, hold for 0.5 min, B: Ethanol+0.2% iso-propyl amine, 10-40% in 8.8 min, hold for 0.5 min; Flow: 3 ml/min; Temp: 50° C.; BPR: 130 bar; Inj. Vol.: 5.0 uL; UV 210 nm; Sample prep.: 2.0 mg/ml Ethanol. Retention times: 2.67 min (2R)-2-benzylpyrrolidine, 3.01 min (2S)-2-benzylpyrrolidine, 3.84 min (S)-phenyl-[(2R)-pyrrolidin-2-yl]methanol, 4.09 min phenyl-[(2R)-pyrrolidin-2-yl]methanone, 4.33 min phenyl-[(2S)-pyrrolidin-2-yl]methanone, 4.55 min (R)-phenyl-[(2S)-pyrrolidin-2-yl]methanol, 4.89 min (S)-phenyl-[(2S)-pyrrolidin-2-yl]methanol, 5.47 min (R)-phenyl-[(2R)-pyrrolidin-2-yl]methanol.

### Purification of (S)-phenyl-[(2R)-pyrrolidin-2-yl]methanol Hydrochloride

A 30 L Reactor was charged with (S)-phenyl((R)-pyrrolidin-2-yl)methanol (1.32 kg, 6.16 mol) with the above mentioned chemical purity of 98% and an enantiomeric excess of 99% and acetonitrile (15.7 kg, 20 l, Eq: -). The resulting suspension was heated to 80° C. until a solution was obtained and the volume was reduced by 1.5 L via destillation. The clear yellow solution was cooled to 0° C. over 3 hr. The resulting suspension was stirred for an additional 1 hr at 0° C. and was then filtered. The white crystals where washed with cold (0° C.) acetonitrile (3.93 kg, 5 l, Eq: -) and dried under reduced pressure to give desired product (1202 g, 91%) with a chemical purity of >99% (see HPLC method bellow) and an enantiomeric excess of >99% (see chiral SFC method bellow)

1H NMR (600 MHz, DMSO-d6) δ ppm 9.42 (br s, 1H), 8.71 (br s, 1H), 7.22-7.46 (m, 5H), 6.09 (br dJ,=3.1 Hz, 1H), 5.03 (br s, 1H), 3.64-3.78 (m, 1H), 3.06-3.21 (m, 2H), 2.04-2.12 (m, 1H), 1.70-1.99 (m, 3H), 1.52-1.64 (m, 1H)

HRMS: (ESI-TOF) calculated for (C₁₁H₁₅NO): 177.1154, found: 177.1161.

HPLC method: Column: XBridge BEH Phenyl, 2.5 um, 100×4.6 mm, Nr: 207; Solvent: A: H2O/ACN, 95/5: 80-45% in 4 min, hold for 1 min, B: ACN: 15-50% in 4 min, hold for 1 min, D: 100 mM Ammonium formate in H2O/ACN (95/5) @ pH9 with NH3: 5% isocratic; Temp: 50° C.; Flow: 1.5 ml/min; Inj. Vol.: 3.5 ul+wash, Starting pressure: 247 bar; Detector: 212 nm, BW: 8 nm, Ref: 360 nm, Ref BW: 50M; Sample Prep: 0.5 mg/ml in H2O/ACN (1/1; v/v)

Chiral SFC method: Chiralcel OZ-3, 150×4.6 mm, Nr: 183; Mobile phases, A: CO₂, 90-60% in 8.8 min, hold for 0.5 min, B: Ethanol+0.2% iso-propyl amine, 10-40% in 8.8 min, hold for 0.5 min; Flow: 3 ml/min; Temp: 50° C.; BPR: 130 bar; Inj. Vol.: 5.0 uL; UV 210 nm; Sample prep.: 2.0 mg/ml Ethanol. Retention times: 2.67 min (2R)-2-benzylpyrrolidine, 3.01 min (2S)-2-benzylpyrrolidine, 3.84 min (S)-phenyl-[(2R)-pyrrolidin-2-yl]methanol, 4.09 min phenyl-[(2R)-pyrrolidin-2-yl]methanone, 4.33 min phenyl-[(2S)-pyrrolidin-2-yl]methanone, 4.55 min (R)-phenyl-[(2S)-pyrrolidin-2-yl]methanol, 4.89 min (S)-phenyl-[(2S)-pyrrolidin-2-yl]methanol, 5.47 min (R)-phenyl-[(2R)-pyrrolidin-2-yl]methanol.

### Example 3

### Preparation of (S)-p-tolyl-[(2R)-pyrrolidin-2-yl]methanol Hydrochloride

Reaction Scheme:

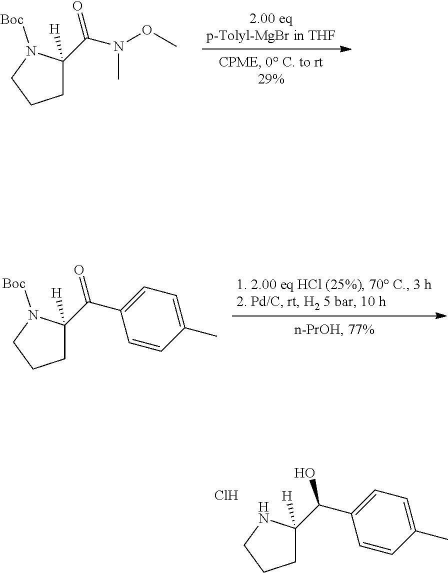 [SMILES: CON(C)C(=O)[C@H]1CCCN1C(=O)OC(C)(C)C ; Cc1ccc(C(=O)[C@H]2CCCN2C(=O)OC(C)(C)C)cc1 ; Cl ; Cc1ccc([C@H](O)[C@H]2CCCN2)cc1]

### a) tert-butyl (2R)-2-(4-methylbenzoyl)pyrrolidine-1-carboxylate

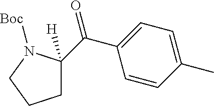 [SMILES: Cc1ccc(C(=O)[C@H]2CCCN2C(=O)OC(C)(C)C)cc1]

A 100-mL-four-necked flask equipped with a magnetic stirrer, argon inlet, thermometer and a syringe pump was charged with (R)-tert-butyl 2-(methoxy(methyl)carbamoyl)pyrrolidine-1-carboxylate (2.5 g, 9 mmol, Eq: 1) in cyclopentyl methyl ether (3 mL). The yellow solution was cooled to 0° C. p-Tolylmagnesium bromide (1M in THF, 18 ml, 18 mmol, Eq: 2) was added dropwise over 30 min maintaining the temperature at 0° C. The resulting light brown-yellow clear solution was stirred for 80 min at 0° C., then warmed to rt over 1 hr and stirred for 3 hr at rt.

The reaction mixture was cooled to 0° C. and carefully quenched with citric acid (25 mL, 1.6M, 40 mmol). The resulting biphasic mixture was allowed to separate and the organic, yellow clear solution was separated and the aqueous layer was extracted with cyclopentyl methyl ether (10 mL). The organic layers were washed with 5% NaHCO₃ (25 mL) and 10% NaCl (20 mL), dried over Na₂SO₄, filtered and evaporated under reduced pressure to give 1.8 g of a clear, orange viscous oil with a chemical purity of 68.9% (see HPLC method below)

The crude material was dissolved in a mixture of i-PrOH/water (1:1, 12 mL) at 60° C. to give an orange clear solution. The solution was cooled to rt over 1 hr and started to crystallize at 30° C. The suspension was cooled to 0° C. and stirred for 2 hr. The crystals were filtered, washed with a mixture of i-PrOH/water (1:1, 5 mL) and dried under reduced pressure. After drying 0.75 g (28.8%) pink crystals were obtained with a chemical purity of 97.5% (see GC method below) and an enantiomeric excess of >99.9% (see chiral SFC method below).

1H NMR (600 MHz, CDCl₃) δ ppm 7.70-8.04 (m, 1H), 5.08-5.36 (m, 1H), 3.34-3.72 (m, 2H), 2.37-2.46 (m, 2H), 2.23-2.34 (m, 1H), 1.84-1.97 (m, 1H), 1.82-1.97 (m, 1H), 1.15-1.54 (m, 7H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 5.79 min tert-butyl (2R)-2-(4-methylbenzoyl)pyrrolidine-1-carboxylate

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 14.64 min tert-butyl (2R)-2-(4-methylbenzoyl)pyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.49 min tert-butyl (2R)-2-(4-methylbenzoyl)pyrrolidine-1-carboxylate, 1.72 min tert-butyl (2S)-2-(4-methylbenzoyl)pyrrolidine-1-carboxylate

### b) (S)-p-tolyl-[(2R)-pyrrolidin-2-yl]methanol Hydrochloride

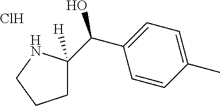

A 25 mL 3-necked flask equipped with a magnetic stirrer and reflux condenser was charged with (R)-tert-butyl 2-(4-methylbenzoyl)pyrrolidine-1-carboxylate (1.2 g, 4.15 mmol, Eq: 1) in 1-propanol (4.8 g, 6 ml, Eq: -). The clear light brown solution was heated to 70° C. and then hydrochloric acid 25% (720 mg, 600 μl, 4.93 mmol, Eq: 1.19) was added dropwise over 1 min. The resulting dark brown solution was stirred at 70° C. for 6 hr. More hydrochloric acid 25% (490 mg, 408 μl, 3.36 mmol, Eq: 0.81) was added dropwise and the solution was stirred at 70° C. for further 2 hr at which point complete disappearance of the starting material was observed. The reaction mixture was cooled to rt and transferred to an autoclave, the flask was rinsed with additional 1-propanol (3.2 g, 4 ml) and this solution was also transferred to the autoclave. After establishing an atmosphere of argon Palladium on Carbon (5.031%, 11 mg, 5.18 μmol, Eq: 0.00125) was added. The autoclave was flushed with H₂ and under stirring the hydrogen pressure was increased to 5 bar at 22° C. After 10 hr the autoclave was ventilated. The reaction mixture was filtered and the filter cake washed with 1-propanol.

The reaction mixture was concentrated under reduced pressure to a viscous oil at which point n-PrOAc (15 mL) was added. The resulting mixture was again concentrated under reduced pressure to a viscous oil at which point more n-PrOAc (10 mL) was added. The resulting suspension was stirred for 1 h at rt, then cooled to 0° C. and stirred for 2.5 hr. The suspension was filtered, and the crystalline white solid was washed with cold (0° C.) n-PrOAc (5 mL). After drying under reduced pressure white crystals (0.73 g, 76.5%) with a chemical purity of 99.0% (see SFC method below) and an enantiomeric purity of 98.9% (see chiral SFC method below) were obtained.

1H NMR (600 MHz, DMSO-d6) δ ppm 8.39-9.59 (m, 2H), 7.23-7.35 (m, 2H), 7.12-7.21 (m, 2H), 5.94-6.10 (m, 1H), 4.91-5.04 (m, 1H), 3.61-3.72 (m, 1H), 3.06-3.20 (m, 2H), 2.29 (s, 3H), 1.53-1.95 (m, 4H)

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 3.74 min (S)-p-tolyl-[(2R)-pyrrolidin-2-yl]methanol

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 4.26 min (S)-p-tolyl-[(2R)-pyrrolidin-2-yl]methanol.

### Example 4

### Preparation of (S)-m-tolyl-[(2R)-pyrrolidin-2-yl]methanol;hydrochloride

Reaction Scheme:

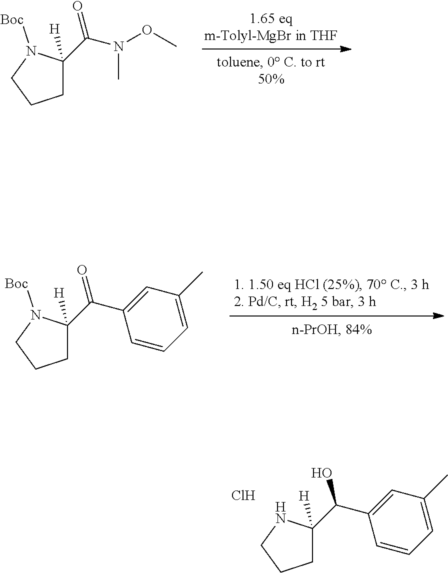 [SMILES: CON(C)C(=O)[C@H]1CCCN1C(=O)OC(C)(C)C ; Cc1cccc(C(=O)[C@H]2CCCN2C(=O)OC(C)(C)C)c1 ; Cl ; Cc1cccc([C@H](O)[C@H]2CCCN2)c1]

### a) tert-butyl (2R)-2-(3-methylbenzoyl)pyrrolidine-1-carboxylate

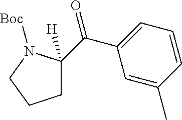 [SMILES: Cc1cccc(C(=O)[C@H]2CCCN2C(=O)OC(C)(C)C)c1]

A 100-mL-four-necked flask equipped with a magnetic stirrer, argon inlet, thermometer and a syringe pump was charged with (R)-tert-butyl 2-(methoxy(methyl)carbamoyl)pyrrolidine-1-carboxylate (2.5 g, 9 mmol, Eq: 1) in toluene (15 mL). The light yellow solution was cooled to 0° C. m-tolylmagnesium bromide (1 M in THF, 13.1 ml, 13.1 mmol, Eq: 1.45) was added dropwise over 30 min maintaining the temperature at 0° C. The resulting light brown clear solution was stirred for 80 min at 0° C., then warmed to rt over 1 hr and stirred for 3 h at rt. The solution was re-cooled to 0° C. and more m-tolylmagnesium bromide (1M in THF, 1.8 ml, 1.8 mmol, Eq: 0.2) was added dropwise over 15 min maintaining the temperature at 0° C., then stirred for 1 h at rt.

The reaction mixture was cooled to 0° C. and carefully quenched with citric acid (25 mL, 1.6M, 40 mmol). The resulting biphasic mixture was allowed to separate and the organic, yellow clear solution was separated and the aqueous layer was extracted with toluene (10 mL). The organic layers were washed with 5% NaHCO₃ (25 mL) and 10% NaCl (20 mL), dried over Na₂SO₄, filtered and evaporated under reduced pressure to give 1.93 g of a red solid with a chemical purity of 66.4% (see HPLC method below)

The crude material was dissolved in a mixture of i-PrOH/water (1:1, 16 mL) at 60° C. to give a red clear solution. The solution was cooled to rt over 1.5 h and started to crystallize at 30° C. The light red/pink suspension was cooled to 0° C. and stirred for 1 hr. The crystals were filtered, washed with a mixture of i-PrOH/water (1:1, 5 mL) and dried under reduced pressure. After drying 1.30 g (49.9%) light pink crystals were obtained with a chemical purity of 97.1% (see GC method below) and an enantiomeric excess of >99.9% (see chiral SFC method below).

1H NMR (600 MHz, CHLOROFORM-d) δ ppm 7.75-7.83 (m, 1H), 7.70-7.83 (m, 1H), 7.33-7.39 (m, 1H), 7.31-7.43 (m, 1H), 5.31-5.36 (m, 1H), 5.17-5.23 (m, 1H), 3.60-3.73 (m, 1H), 3.44-3.59 (m, 1H), 2.37-2.46 (m, 3H), 2.23-2.36 (m, 1H), 1.85-2.01 (m, 3H), 1.44-1.51 (m, 4H), 1.28 (s, 5H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 5.78 min tert-butyl (2R)-2-(3-methylbenzoyl)pyrrolidine-1-carboxylate

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 14.50 min tert-butyl (2R)-2-(3-methylbenzoyl)pyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.33 min tert-butyl (2R)-2-(3-methylbenzoyl)pyrrolidine-1-carboxylate, 1.49 min tert-butyl (2S)-2-(3-methylbenzoyl)pyrrolidine-1-carboxylate

### b) (S)-m-tolyl-[(2R)-pyrrolidin-2-yl]methanol Hydrochloride

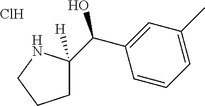

A 25 mL 3-necked flask equipped with a magnetic stirrer and reflux condenser was charged with (R)-tert-butyl 2-(3-methylbenzoyl)pyrrolidine-1-carboxylate (1 g, 3.46 mmol, Eq: 1) in 1-propanol (4 g, 5 ml, Eq: -). The clear solution was heated to 70° C. and then hydrochloric acid 25% (756 mg, 630 μl, 5.18 mmol, Eq: 1.5) was added dropwise over 1 min. The clear solution was stirred at 70° C. for 3 hr at which point complete disappearance of the starting material was observed. The reaction mixture was cooled to rt and transferred to an autoclave, the flask was rinsed with additional 1-propanol (4 g, 5 ml) and this solution was also transferred to the autoclave. After establishing an atmosphere of argon Palladium on Carbon (5.031%, 18.3 mg, 8.65 μmol, Eq: 0.0025) was added. The autoclave was flushed with H₂ and under stirring the hydrogen pressure was increased to 5 bar at 22° C. After 3 hr the autoclave was ventilated. The reaction mixture was filtered and the filter cake washed with 1-propanol.

The reaction mixture was concentrated under reduced pressure to a viscous oil at which point n-PrOAc (15 mL) was added. The resulting mixture was again concentrated and suspended in n-PrOAc (15 mL), the resulting suspension was again concentrated under reduced pressure. To the residue n-PrOAc (10 mL) was added. The resulting suspension was stirred for 30 min at rt, then cooled to 0° C. and stirred for 2 hr. The suspension was filtered, and the crystalline white solid was washed with cold (0° C.) n-PrOAc (5 mL). After drying under reduced pressure white crystals (0.68 g, 84.1%) with a chemical purity of 97.4% (see SFC method below) and an enantiomeric purity of >99.9% (see chiral SFC method below) were obtained.

1H NMR (600 MHz, DMSO-d6) δ ppm 9.16-9.67 (m, 1H), 8.37-8.88 (m, 1H), 7.24-7.28 (m, 1H), 7.21 (s, 1H), 7.19 (dJ,=7.7 Hz, 1H), 7.11 (d, J=7.5 Hz, 1H), 5.89-6.16 (m, 1H), 4.99 (br d, J=3.3 Hz, 1H), 3.63-3.74 (m, 1H), 3.08-3.21 (m, 2H), 2.32 (s, 3H), 1.56-1.93 (m, 4H).

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 3.65 min (S)-m-tolyl-[(2R)-pyrrolidin-2-yl]methanol hydrochloride

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 3.92 min (S)-m-tolyl-[(2R)-pyrrolidin-2-yl]methanol hydrochloride.

### Example 5

### Preparation of (S)-(4-tert-butylphenyl)-[(2R)-pyrrolidin-2-yl]methanol Hydrochloride

Reaction Scheme:

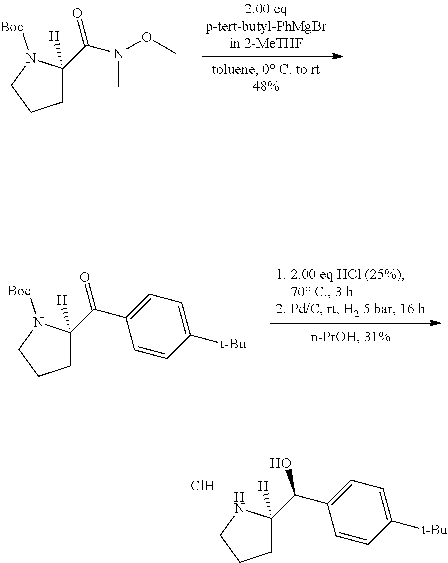 [SMILES: CON(C)C(=O)[C@H]1CCCN1C(=O)OC(C)(C)C ; CC(C)(C)OC(=O)N1CCC[C@@H]1C(=O)c1ccc(C(C)(C)C)cc1 ; Cl ; CC(C)(C)c1ccc([C@H](O)[C@H]2CCCN2)cc1]

### a) tert-butyl (2R)-2-(4-tert-butylbenzoyl)pyrrolidine-1-carboxylate

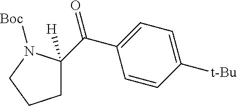 [SMILES: CC(C)(C)OC(=O)N1CCC[C@@H]1C(=O)c1ccc(C(C)(C)C)cc1]

A 100-mL-four-necked flask equipped with a magnetic stirrer, argon inlet, thermometer and a syringe pump was charged with (R)-tert-butyl 2-(methoxy(methyl)carbamoyl)pyrrolidine-1-carboxylate (4 g, 15.1 mmol, Eq: 1) in toluene (5 mL). The light yellow solution was cooled to 0° C. (4-(tert-Butyl)phenyl)magnesium bromide (0.5M in 2-MeTHF, 60.5 ml, 30.3 mmol, Eq: 2) was added dropwise over 30 min maintaining the temperature at 0° C. The resulting light orange clear solution was stirred for 60 min at 0° C., then warmed to rt and stirred for 17 hr at rt.

The reaction mixture was cooled to 0° C. and carefully quenched with citric acid (30 mL, 1.6M, 48 mmol). The resulting biphasic mixture was allowed to separate and the organic, orange clear solution was separated and the aqueous layer was extracted with toluene (15 mL). The organic layers were washed with 5% NaHCO₃ (60 mL) and 10% NaCl (50 mL), dried over Na₂SO₄, filtered and evaporated under reduced pressure to give 5.91 g of an orange solid with a chemical purity of 67.8% (see HPLC method below)

The crude material was dissolved in a mixture of i-PrOH/water (1:1, 42 mL) at 70° C. to give an orange clear solution. The solution was cooled to rt over 30 min and started to crystallize at 30° C. The suspension was stirred for 2.5 hr at rt. The crystals (off-white) were filtered, washed with a mixture of i-PrOH/water (1:1, 5 mL) and dried under reduced pressure. After drying 3.0 g (47.9%) off-white crystals were obtained with a chemical purity of 80.7% (see GC method below) and an enantiomeric excess of >99.9% (see chiral SFC method below).

1H NMR (600 MHz, CHLOROFORM-d) δ ppm 7.84-7.97 (m, 2H), 7.44-7.50 (m, 2H), 5.13-5.36 (m, 1H), 3.40-3.78 (m, 2H), 2.24-2.35 (m, 1H), 1.97 (br s, 2H), 1.85-1.97 (m, 1H), 1.44-1.48 (m, 4H), 1.32-1.35 (m, 9H), 1.27 (s, 4H), 1.25-1.28 (m, 1H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 6.76 min tert-butyl (2R)-2-(4-tert-butylbenzoyl)pyrrolidine-1-carboxylate

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 15.64 min tert-butyl (2R)-2-(4-tert-butylbenzoyl)pyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.53 min tert-butyl (2R)-2-(4-tert-butylbenzoyl)pyrrolidine-1-carboxylate, 1.63 min tert-butyl (2S)-2-(4-tert-butylbenzoyl)pyrrolidine-1-carboxylate

### b) (S)-(4-tert-butylphenyl)-[(2R)-pyrrolidin-2-yl]methanol Hydrochloride

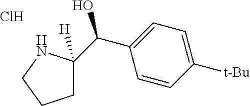 [SMILES: Cl ; CC(C)(C)c1ccc([C@H](O)[C@H]2CCCN2)cc1]

A 25 mL 3-necked flask equipped with a magnetic stirrer and reflux condenser was charged with (R)-tert-butyl 2-(4-(tert-butyl)benzoyl)pyrrolidine-1-carboxylate (1.5 g, 3.62 mmol, Eq: 1) in 1-propanol (6 g, 7.5 ml, Eq: -). The clear light brown solution was heated to 70° C. and then hydrochloric acid 25% (628 mg, 524 μl, 4.31 mmol, Eq: 1.19) was added dropwise over 1 min. The brown solution was stirred at 70° C. for 6 hr. More hydrochloric acid 25% (428 mg, 356 μl, 2.93 mmol, Eq: 0.81) was added dropwise and the solution was stirred at 70° C. for another 2 hr at which point complete disappearance of the starting material was observed. The reaction mixture was cooled to rt and transferred to an autoclave, the flask was rinsed with additional 1-propanol (4 g, 5 ml, Eq: -) and this solution was also transferred to the autoclave. After establishing an atmosphere of argon Palladium on Carbon (5.031%, 9.57 mg, 4.52 μmol, Eq: 0.00125) was added. The autoclave was flushed with H₂ and under stirring the hydrogen pressure was increased to 5 bar at 22° C. After 16 hr the autoclave was ventilated. The reaction mixture was filtered and the filter cake washed with 1-propanol.

The reaction mixture was concentrated under reduced pressure to a viscous oil at which point n-PrOAc (15 mL) was added. The resulting mixture was again concentrated under reduced pressure. n-PrOAc (10 mL) was added and the resulting suspension was stirred for 1 h at rt, then cooled to 0° C. and stirred for 2.5 hr. The suspension was filtered, and the crystalline off-white solid was washed with cold (0° C.) n-PrOAc (5 mL). After drying under reduced pressure off-white crystals (0.31 g, 31.0%) with a chemical purity of 97.5% (see SFC method below) and an enantiomeric purity of >99.9% (see chiral SFC method below) were obtained.

1H NMR (600 MHz, DMSO-d6) δ ppm 8.51-9.55 (m, 2H), 7.37-7.41 (m, 2H), 7.30-7.35 (m, 2H), 5.93-6.06 (m, 1H), 4.94-5.01 (m, 1H), 3.68 (br d, J=4.1 Hz, 1H), 3.08-3.20 (m, 2H), 1.56-1.97 (m, 4H), 1.26-1.29 (m, 9H)

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 3.56 min (S)-(4-tert-butylphenyl)-[(2R)-pyrrolidin-2-yl]methanol hydrochloride

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 4.11 min (S)-(4-tert-butylphenyl)-[(2R)-pyrrolidin-2-yl]methanol hydrochloride.

### Example 6

### Preparation of (S)-2-naphthyl-[(2R)-pyrrolidin-2-yl]methanol Hydrochloride

Reaction Scheme:

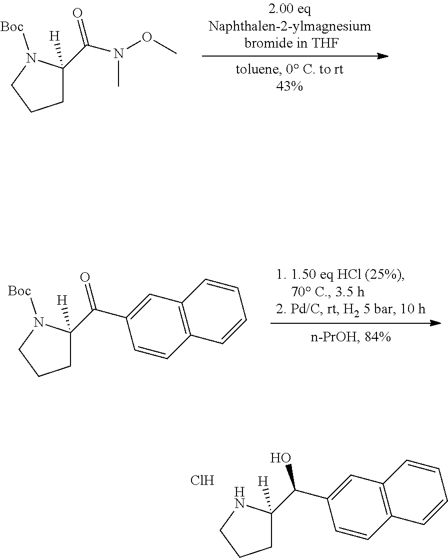 [SMILES: CON(C)C(=O)[C@H]1CCCN1C(=O)OC(C)(C)C ; CC(C)(C)OC(=O)N1CCC[C@@H]1C(=O)c1ccc2ccccc2c1 ; Cl ; O[C@@H](c1ccc2ccccc2c1)[C@H]1CCCN1]

### a) tert-butyl (2R)-2-(naphthalene-2-carbonyl)pyrrolidine-1-carboxylate

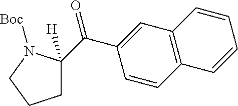 [SMILES: CC(C)(C)OC(=O)N1CCC[C@@H]1C(=O)c1ccc2ccccc2c1]

A 100-mL-four-necked flask equipped with a magnetic stirrer, argon inlet, thermometer and a syringe pump was charged with (R)-tert-butyl 2-(methoxy(methyl)carbamoyl)pyrrolidine-1-carboxylate (3 g, 11.3 mmol, Eq: 1) in toluene (4 mL). The light yellow solution was cooled to 0° C. Naphthalen-2-ylmagnesium bromide (0.5M in THF, 45.4 ml, 22.7 mmol, Eq: 2) was added dropwise over 30 min maintaining the temperature at 0° C. The resulting light brown clear solution was stirred for 1 hr at 0° C., then warmed to rt and stirred for 16 hr at rt. The solution became turbid.

The reaction mixture was cooled to 0° C. and carefully quenched with citric acid (25 mL, 1.6M, 40 mmol). The resulting biphasic mixture was allowed to separate and the organic, yellow clear solution was separated and the aqueous layer was extracted with toluene (15 mL). The organic layers were washed with 5% NaHCO₃ (45 mL) and 10% NaCl (30 mL), dried over Na₂SO₄, filtered and evaporated under reduced pressure to give 4.65 g of an orange solid with a chemical purity of 45.7% (see HPLC method below)

The crude material was dissolved in a mixture of i-PrOH/water (1:1, 36 mL) at 80° C. to give an orange clear solution. The solution was cooled to rt over 30 min and started to crystallize. The suspension was cooled to 0° C. and stirred for 2 hr. The crystals were filtered and dried under reduced pressure. After drying 2.1 g brown crystals were obtained with a chemical purity of 55.3% (see HPLC method below). The brown crystals were further purified via flash chromatography (SiO₂, 40 g, EtOAc:Heptane 1:9 to 1:1). After drying for 2 hr under reduced pressure, 1.63 g (42.6%) light brown solid was obtained with a chemical purity of 94.6% (see GC method below) and an enantiomeric excess of >99.9% (see chiral SFC method below).

1H NMR (600 MHz, CHLOROFORM-d) δ ppm 8.46-8.55 (m, 1H), 5.37 (dd, J=9.0, 3.8 Hz, 1H), 3.43-3.83 (m, 2H), 2.31-2.46 (m, 1H), 1.93-2.04 (m, 1H), 1.90-2.04 (m, 2H), 1.20-1.51 (m, 9H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 5.5 min tert-butyl (2R)-2-(naphthalene-2-carbonyl)pyrrolidine-1-carboxylate.

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H2); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 17.26 min tert-butyl (2R)-2-(naphthalene-2-carbonyl)pyrrolidine-1-carboxylate.

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 2.12 min tert-butyl (2R)-2-(naphthalene-2-carbonyl)pyrrolidine-1-carboxylate, 5.53 min tert-butyl (2S)-2-(naphthalene-2-carbonyl)pyrrolidine-1-carboxylate.

### b) (S)-2-naphthyl-[(2R)-pyrrolidin-2-yl]methanol Hydrochloride

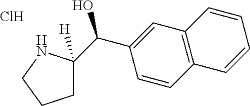 [SMILES: Cl ; O[C@@H](c1ccc2ccccc2c1)[C@H]1CCCN1]

A 25 mL 3-necked flask equipped with a magnetic stirrer and a reflux condenser was charged with (R)-tert-butyl 2-(2-naphthoyl)pyrrolidine-1-carboxylate (0.8 g, 2.37 mmol, Eq: 1) in 1-propanol (3.2 g, 4 ml, Eq: -). The clear yellow solution was heated to 70° C. and then hydrochloric acid 25% (519 mg, 433 μl, 3.56 mmol, Eq: 1.5) was added dropwise over 1 min. The clear light yellow solution was stirred at 70° C. for 3.5 hr at which point complete disappearance of the starting material was observed. The reaction mixture was cooled to rt and transferred to an autoclave, the flask was rinsed with additional 1-propanol (3.2 g, 4 ml, Eq: -) and this solution was also transferred to the autoclave. After establishing an atmosphere of argon Palladium on Carbon (5.031%, 12.5 mg, 5.93 μmol, Eq: 0.0025) was added. The autoclave was flushed with H₂ and under stirring the hydrogen pressure was increased to 5 bar at 22° C. After 10 hr the autoclave was ventilated. The reaction mixture was filtered and the filter cake washed with 1-propanol.

The reaction mixture was concentrated under reduced pressure to a viscous oil at which point n-PrOAc (15 mL) was added. The resulting mixture was again concentrated under reduced pressure. n-PrOAc (15 mL) was added and the resulting suspension was stirred for 30 min at rt, then cooled to 0° C. and stirred for 2 hr. The suspension was filtered, and the crystalline light brown solid was washed with cold (0° C.) n-PrOAc (5 mL). After drying under reduced pressure light brown crystals (0.55 g, 83.9%) with a chemical purity of 95.4% (see SFC method below) and an enantiomeric purity of 95.0% (see chiral SFC method below) were obtained.

1H NMR (600 MHz, DMSO-d6) δ ppm 8.52-9.62 (m, 2H), 7.83-8.01 (m, 4H), 7.42-7.62 (m, 3H), 6.25 (br dJ,=3.3 Hz, 1H), 5.19 (br s, 1H), 3.82 (br d, J=4.2 Hz, 1H), 3.04-3.25 (m, 2H), 1.45-1.97 (m, 4H)

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 4.60 min (S)-2-naphthyl-[(2R)-pyrrolidin-2-yl]methanol hydrochloride

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 5.30 min (S)-2-naphthyl-[(2R)-pyrrolidin-2-yl]methanol hydrochloride.

### Example 7

### Preparation of (S)-(3,5-difluorophenyl)-[(2R)-pyrrolidin-2-yl]methanol Hydrochloride

Reaction Scheme:

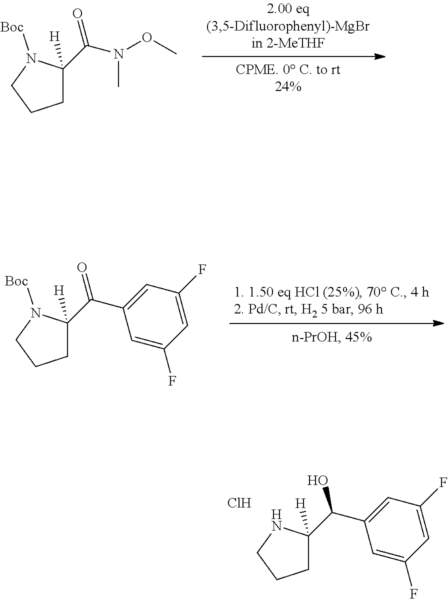 [SMILES: CON(C)C(=O)[C@H]1CCCN1C(=O)OC(C)(C)C ; CC(C)(C)OC(=O)N1CCC[C@@H]1C(=O)c1cc(F)cc(F)c1 ; Cl ; O[C@@H](c1cc(F)cc(F)c1)[C@H]1CCCN1]

### a) tert-butyl (2R)-2-(3,5-difluorobenzoyl)pyrrolidine-1-carboxylate

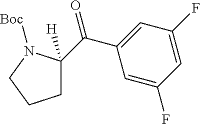 [SMILES: CC(C)(C)OC(=O)N1CCC[C@@H]1C(=O)c1cc(F)cc(F)c1]

A 100-mL-four-necked flask equipped with a magnetic stirrer, argon inlet, thermometer and a syringe pump was charged with (R)-tert-butyl 2-(methoxy(methyl)carbamoyl)pyrrolidine-1-carboxylate (2.2 g, 7.92 mmol, Eq: 1) in cyclopentyl methyl ether (3 mL). The light yellow solution was cooled to 0° C. (3,5-Difluorophenyl)magnesium bromide (0.5 M in 2-MeTHF, 31.7 ml, 15.8 mmol, Eq: 2) was added dropwise over 30 min maintaining the temperature at 0° C. The resulting light brown-yellow clear solution was stirred for 80 min at 0° C., then warmed to rt over 1 h and stirred for 19 h at rt. After 19 h at rt the clear solution became turbid.

The reaction mixture was cooled to 0° C. and carefully quenched with citric acid (25 mL, 1.6M, 40 mmol). The resulting biphasic mixture was allowed to separate and the organic, yellow clear solution was separated and the aqueous layer was extracted with cyclopentyl methyl ether (10 mL). The organic layers were washed twice with 5% NaHCO₃ (25 mL) and 10% NaCl (20 mL), dried over Na₂SO₄, filtered and evaporated under reduced pressure to give 1.93 g of a clear, yellow oil with a chemical purity of 40.7% (see HPLC method below)

The crude material was dissolved in a mixture of i-PrOH/water (1:1, 6 mL) at 60° C. to give a yellow clear solution. The solution was cooled to rt over 30 min and started to crystallize at 30° C. The yellow suspension was cooled to 0° C. and stirred for 1 hr. The crystals were filtered, washed with a mixture of i-PrOH/water (1:1, 2 mL) and dried under reduced pressure. After drying 1.22 g (24%) light yellow crystals were obtained with a chemical purity of 98.8% (see GC method below) and an enantiomeric excess of >99.9% (see chiral SFC method below).

1H NMR (600 MHz, CDCL3) δ ppm 7.40-7.54 (m, 2H), 6.95-7.10 (m, 1H), 4.95-5.24 (m, 1H), 3.36-3.69 (m, 2H), 2.14-2.38 (m, 1H), 1.79-2.03 (m, 2H), 1.78-2.02 (m, 1H), 1.16-1.52 (m, 9H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 5.92 min tert-butyl (2R)-2-(3,5-difluorobenzoyl)pyrrolidine-1-carboxylate GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 13.47 min tert-butyl (2R)-2-(3,5-difluorobenzoyl)pyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.17 min tert-butyl (2R)-2-(3,5-difluorobenzoyl)pyrrolidine-1-carboxylate, 1.64 min tert-butyl (2S)-2-(3,5-difluorobenzoyl)pyrrolidine-1-carboxylate.

### b) (S)-(3,5-difluorophenyl)-[(2R)-pyrrolidin-2-yl]methanol Hydrochloride

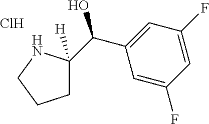 [SMILES: Cl ; O[C@@H](c1cc(F)cc(F)c1)[C@H]1CCCN1]

A 25 mL 3-necked flask equipped with a magnetic stirrer and a reflux condenser was charged with (R)-tert-butyl 2-(3,5-difluorobenzoyl)pyrrolidine-1-carboxylate (1 g, 3.12 mmol, Eq: 1) in 1-propanol (4 g, 5 ml, Eq: -). The clear solution was heated to 70° C. and then hydrochloric acid 25% (683 mg, 569 μl, 4.68 mmol, Eq: 1.5) was added dropwise over 1 min.

The clear light yellow solution was stirred at 70° C. for 4 hr at which point complete disappearance of the starting material was observed. The reaction mixture was cooled to rt and transferred to an autoclave, the flask was rinsed with additional 1-propanol (4 g, 5 ml, Eq: -) and this solution was also transferred to the autoclave. After establishing an atmosphere of argon Palladium on Carbon (5.031%, 16.5 mg, 7.8 μmol, Eq: 0.0025) was added. The autoclave was flushed with H₂ and under stirring the hydrogen pressure was increased to 5 bar at 22° C. After 96 hr the autoclave was ventilated. The reaction mixture was filtered and the filter cake washed with 1-propanol.

The reaction mixture was concentrated under reduced pressure to a viscous oil at which point n-PrOAc (10 mL) was added. The resulting mixture was again concentrated under reduced pressure. n-PrOAc (10 mL) was added and the resulting suspension was stirred for 30 min at rt, then cooled to 0° C. and stirred for 2 hr. The suspension was filtered, and the crystalline white solid was washed with cold (0° C.) n-PrOAc (5 mL). After drying under reduced pressure white crystals (360 mg, 44.5%) with a chemical purity of 96.4% (see SFC method below) and an enantiomeric purity of >99.9% (see chiral SFC method below) were obtained.

1H NMR (600 MHz, DMSO-d6) δ ppm 9.22-9.50 (m, 1H), 8.72-9.04 (m, 1H), 7.15-7.21 (m, 1H), 7.14-7.18 (m, 2H), 6.11-6.46 (m, 1H), 5.07 (br s, 1H), 3.69-3.80 (m, 1H), 3.10-3.21 (m, 2H), 1.87-1.97 (m, 1H), 1.73-1.84 (m, 1H), 1.73-1.84 (m, 1H), 1.62-1.71 (m, 1H), 0.80-0.85 (m, 1H)

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 3.63 min (S)-(3,5-difluorophenyl)-[(2R)-pyrrolidin-2-yl]methanol hydrochloride

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 2.30 min (S)-(3,5-difluorophenyl)-[(2R)-pyrrolidin-2-yl]methanol hydrochloride.

### Example 8

### Preparation of (S)-(3,5-dimethoxyphenyl)-[(2R)-pyrrolidin-2-yl]methanol Hydrochloride

Reaction Scheme:

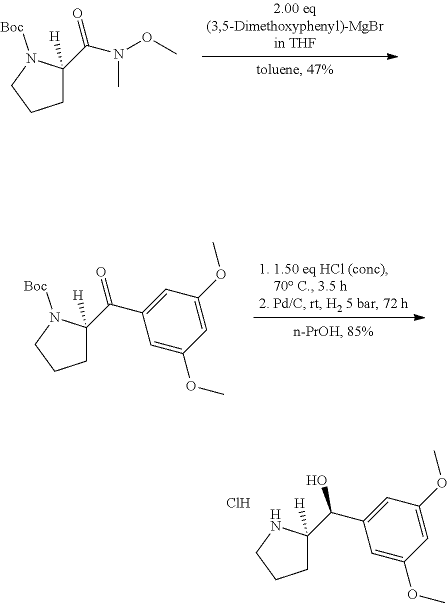 [SMILES: CON(C)C(=O)[C@H]1CCCN1C(=O)OC(C)(C)C ; COc1cc(OC)cc(C(=O)[C@H]2CCCN2C(=O)OC(C)(C)C)c1 ; Cl ; COc1cc(OC)cc([C@H](O)[C@H]2CCCN2)c1]

### a) tert-butyl (2R)-2-(3,5-dimethoxybenzoyl)pyrrolidine-1-carboxylate

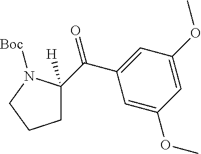 [SMILES: COc1cc(OC)cc(C(=O)[C@H]2CCCN2C(=O)OC(C)(C)C)c1]

A 100-mL-four-necked flask equipped with a magnetic stirrer, argon inlet, a thermometer and a syringe pump was charged with (R)-tert-butyl 2-(methoxy(methyl)carbamoyl)pyrrolidine-1-carboxylate (4 g, 15.1 mmol, Eq: 1) in toluene (5 mL). The light yellow solution was cooled to 0° C. (3,5-Dimethoxyphenyl) magnesium bromide (0.5M in THF, 60.5 ml, 30.3 mmol, Eq: 2) was added dropwise over 30 min maintaining the temperature at 0° C. The resulting light brown clear solution was stirred for 60 min at 0° C., then warmed to rt and stirred for 3 hr at rt.

The reaction mixture was cooled to 0° C. and carefully quenched with citric acid (30 mL, 1.6M, 48 mmol). The resulting biphasic mixture was allowed to separate and the organic, yellow clear solution was separated and the aqueous layer was extracted with toluene (15 mL). The organic layers were washed with 5% NaHCO₃ (60 mL) and 10% NaCl (40 mL), dried over Na₂SO₄, filtered and evaporated under reduced pressure to give 5.74 g of an orange oil with a chemical purity of 46.0% (see HPLC method below)

The crude material was purified via flash chromatography (SiO₂, 80 g, EtOAc:Heptane 1:9 to 1:1). After drying under reduced pressure, 2.56 g (46.8%) of a light yellow viscous oil was obtained with a chemical purity of 95.1% (see GC method below) and an enantiomeric excess of >99.9% (see chiral SFC method below).

1H NMR (600 MHz, CHLOROFORM-d) δ ppm 7.02-7.15 (m, 2H), 6.61-6.69 (m, 1H), 5.08-5.29 (m, 1H), 3.79-3.86 (m, 6H), 3.41-3.70 (m, 2H), 1.22-1.48 (m, 9H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 5.64 min tert-butyl (2R)-2-(3,5-dimethoxybenzoyl)pyrrolidine-1-carboxylate

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 16.16 min tert-butyl (2R)-2-(3,5-dimethoxybenzoyl)pyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.40 min tert-butyl (2R)-2-(3,5-dimethoxybenzoyl)pyrrolidine-1-carboxylate, 2.27 min tert-butyl (2S)-2-(3,5-dimethoxybenzoyl)pyrrolidine-1-carboxylate

### b) (S)-(3,5-dimethoxyphenyl)-[(2R)-pyrrolidin-2-yl]methanol Hydrochloride

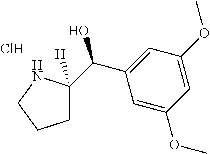 [SMILES: Cl ; COc1cc(OC)cc([C@H](O)[C@H]2CCCN2)c1]

A 25 mL 3-necked flask equipped with a magnetic stirrer and reflux condenser was charged with (R)-tert-butyl 2-(3,5-dimethoxybenzoyl)pyrrolidine-1-carboxylate (1.2 g, 3.5 mmol, Eq: 1) in 1-propanol (4.8 g, 6 ml, Eq: -). The clear solution was heated to 70° C. and then hydrochloric acid 25% (765 mg, 637 μl, 5.24 mmol, Eq: 1.5) was added dropwise over 1 min. The clear solution was stirred at 70° C. for 3.5 hr at which point complete disappearance of the starting material was observed. The reaction mixture was cooled to rt and transferred to an autoclave, the flask was rinsed with additional 1-propanol (3.2 g, 4 ml, Eq: -) and this solution was also transferred to the autoclave. After establishing an atmosphere of argon Palladium on Carbon (5.031%, 18.5 mg, 8.75 μmol, Eq: 0.0025) was added. The autoclave was flushed with H₂ and under stirring the hydrogen pressure was increased to 5 bar at 22° C. After 72 hr the autoclave was ventilated. The reaction mixture was filtered and the filter cake washed with 1-propanol.

The reaction mixture was concentrated under reduced pressure to a viscous oil at which point n-PrOAc (15 mL) was added. The resulting mixture was again concentrated under reduced pressure. n-PrOAc (15 mL) was added and the resulting suspension was stirred for 30 min at rt, then cooled to 0° C. and stirred for 2 hr. The suspension was filtered, and the crystalline white solid was washed with cold (0° C.) n-PrOAc (5 mL). After drying under reduced pressure white crystals (0.82 g, 85.1%) with a chemical purity of 99.4% (see SFC method below) and an enantiomeric purity of 99.3% (see chiral SFC method below) were obtained.

1H NMR (600 MHz, DMSO-d6) δ ppm 8.51-9.45 (m, 2H), 6.58 (d, J=2.3 Hz, 2H), 6.36-6.45 (m, 1H), 5.97-6.12 (m, 1H), 4.82-5.01 (m, 1H), 3.74 (s, 6H), 3.64-3.72 (m, 1H), 3.05-3.19 (m, 2H), 1.60-1.96 (m, 4H)

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 3.87 min (S)-(3,5-dimethoxyphenyl)-[(2R)-pyrrolidin-2-yl]methanol hydrochloride

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 4.17 min (S)-(3,5-dimethoxyphenyl)-[(2R)-pyrrolidin-2-yl]methanol hydrochloride.

### Example 9

### Preparation of (S)-[(2R)-pyrrolidin-2-yl]-(3,4,5-trifluorophenyl)methanol Hydrochloride

Reaction Scheme:

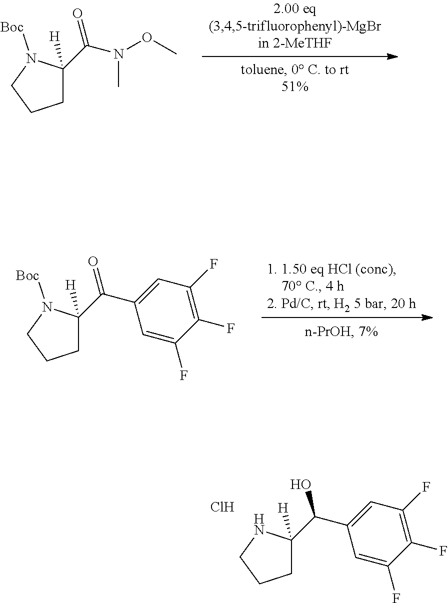 [SMILES: CON(C)C(=O)[C@H]1CCCN1C(=O)OC(C)(C)C ; CC(C)(C)OC(=O)N1CCC[C@@H]1C(=O)c1cc(F)c(F)c(F)c1 ; Cl ; O[C@@H](c1cc(F)c(F)c(F)c1)[C@H]1CCCN1]

### a) tert-butyl (2R)-2-(3,4,5-trifluorobenzoyl)pyrrolidine-1-carboxylate

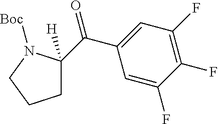 [SMILES: CC(C)(C)OC(=O)N1CCC[C@@H]1C(=O)c1cc(F)c(F)c(F)c1]

A 100-mL-four-necked flask equipped with a magnetic stirrer, argon inlet, thermometer and a syringe pump was charged with (R)-tert-butyl 2-(methoxy(methyl)carbamoyl)pyrrolidine-1-carboxylate (2.5 g, 9.46 mmol, Eq: 1) in toluene (3 mL). The light yellow solution was cooled 10 to 0° C. (3,4,5-trifluorophenyl)magnesium bromide (0.5M in 2-MeTHF, 37.8 ml, 18.9 mmol, Eq: 2) was added dropwise over 30 min maintaining the temperature at 0° C. The resulting light brown clear solution was stirred for 60 min at 0° C., then warmed to rt over 1 hr and stirred for 19 hr at rt. The brown solution became turbid.

The reaction mixture was cooled to 0° C. and carefully quenched with citric acid (25 mL, 1.6M, 40 mmol). The resulting biphasic mixture was allowed to separate and the organic, yellow clear solution was separated and the aqueous layer was extracted with toluene (10 mL). The organic layers were washed with 5% NaHCO₃ (30 mL) and 10% NaCl (30 mL), dried over Na₂SO₄, filtered and evaporated under reduced pressure to give 2.78 g of a yellow oil with a chemical purity of 50.0% (see HPLC method below)

The crude material was purified via flash chromatography (SiO₂, 80 g, EtOAc:Heptane 1:9 to 1:1). After drying under reduced pressure, 1.66 g (51.4%) of colourless viscous oil was obtained with a chemical purity of 96.5% (see GC method below) and an enantiomeric excess of >99.9% (see chiral SFC method below).

1H NMR (600 MHz, CHLOROFORM-d) δ ppm 7.63 (dt, J=16.0, 7.1 Hz, 2H), 4.89-5.31 (m, 1H), 3.42-3.74 (m, 2H), 2.22-2.39 (m, 1H), 1.81-2.01 (m, 3H), 1.18-1.49 (m, 9H)

HPLC method: Column: Waters XBridge C8 2.5 um, 4.6×100 mm Columen XP (PN:186006051); Mobile phases, A: H₂O 95:5 ACN=80-10% in 6 min, hold 2 min, B: ACN=10-80% in 6 min, hold 2 min, C: Wasser+0.5% TFA=10% isocratic; Flow: 1.500 mL/min; Temp.: 45° C.; DAD: 210 nm (BW: 4 nm), Inj Volume: 2.000 μl; Sample Preparation: 2-3 drops reaction mixture quenched with 2 ml MeOH/water and filtrated. Retention times: 3.9 min (R)-tert-butyl 2-(methoxy(methyl)carbamoyl)pyrrolidine-1-carboxylate, 6.15 min tert-butyl (2R)-2-(3,4,5-trifluorobenzoyl)pyrrolidine-1-carboxylate

GC method: Column: HP-5, 30 m×0.32 mm ID, 0.25 um; Temp: 50° C. to 150° C., 10° C./min, 150° C. to 250° C., 20° C./min, at 250° C. hold up 3 min; Injector: 200° C.; Detector: 280° C.; Inj. Vol.: 1 μl; Pressure: 44 kPa, (H₂); Flow: 2.7 ml/min; Average Velocity: 50 cm/sec; FID: Air: 400 ml/min; H₂: 30 ml/min; Makeup Flow: 30 ml/min; Split ratio: 5:1; Split flow 13.5 ml/min; Sample Preparation: 1.0 mg/mL EtOAc. Retention times: 12.41 min (R)-tert-butyl 2-(methoxy(methyl)carbamoyl)pyrrolidine-1-carboxylate, 13.47 min tert-butyl (2R)-2-(3,4,5-trifluorobenzoyl)pyrrolidine-1-carboxylate

Chiral SFC method: Column: Chiralpak AD-3, 3 um, 4.6 mm×250 mm, Nr. 890; Mobile phases, A: CO₂, 85%, B: MeOH+0.2% TFA, 15%; Flow: 3.0 mL/min isocratic; Temp: 40° C., BPR: 130 bar; Inj. Vol.: 3.0 uL; UV 240 nm, Sample prep.: 1.5 mg/ml methanol. Retention times: 1.17 min tert-butyl (2R)-2-(3,4,5-trifluorobenzoyl)pyrrolidine-1-carboxylate, 1.42 min tert-butyl (2S)-2-(3,4,5-trifluorobenzoyl)pyrrolidine-1-carboxylate7

### b) (S)-[(2R)-pyrrolidin-2-yl]-(3,4,5-trifluorophenyl)methanol Hydrochloride

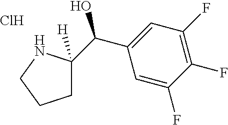 [SMILES: Cl ; O[C@@H](c1cc(F)c(F)c(F)c1)[C@H]1CCCN1]

A 25 mL 3-necked flask equipped with a magnetic stirrer and cooler was charged with (R)-tert-butyl 2-(3,4,5-trifluorobenzoyl)pyrrolidine-1-carboxylate (1.2 g, 3.52 mmol, Eq: 1) in 1-propanol (4.8 g, 6 ml, Eq: -). The clear light yellow solution was heated to 70° C. and then hydrochloric acid 25% (769 mg, 641 μl, 5.27 mmol, Eq: 1.5) was added dropwise over 1 min.

The clear light yellow solution was stirred at 70° C. for 4 hr at which point complete disappearance of the starting material was observed. The reaction mixture was cooled to rt and transferred to an autoclave, the flask was rinsed with additional 1-propanol (4.8 g, 6 ml, Eq: -) and this solution was also transferred to the autoclave. After establishing an atmosphere of argon Palladium on Carbon (5.031%, 18.6 mg, 8.8 μmol, Eq: 0.0025) was added. The autoclave was flushed with H₂ and under stirring the hydrogen pressure was increased to 5 bar at 22° C. After 20 hr the autoclave was ventilated. The reaction mixture was filtered and the filter cake washed with 1-propanol.

The reaction mixture was concentrated under reduced pressure to a viscous oil at which point n-PrOAc (15 mL) was added. The resulting mixture was again concentrated under reduced pressure. n-PrOAc (10 mL) was added and the resulting suspension was stirred for 30 min at rt, then cooled to 0° C. and stirred for 2.5 hr. The suspension was filtered, and the crystalline white solid was washed with cold (0° C.) n-PrOAc (5 mL). After drying under reduced pressure white crystals (70 mg, 6.65%) with a chemical purity of 89.5% (see SFC method below) and an enantiomeric purity of 94.2% (see chiral SFC method below) were obtained.

1H NMR (600 MHz, DMSO-d6) δ ppm 9.17-9.43 (m, 1H), 8.75-8.96 (m, 1H), 7.34-7.41 (m, 2H), 6.28-6.49 (m, 1H), 4.94-5.10 (m, 1H), 3.65-3.77 (m, 1H), 3.10-3.22 (m, 2H), 1.89-1.95 (m, 1H), 1.73-1.81 (m, 1H), 1.73-1.80 (m, 1H), 1.68 (dq, J=11.5, 7.5 Hz, 1H)

SFC method: Acquity UPC2 Torus DEA, 3 um, 4.6 mm×100 mm, Nr. 122; Mobile phases, A: CO₂, 97%-65% in 6 min, B: EtOH+0.2% IPAm, 3-35% in 6 min; Flow: 2.5 mL/min; Temp: 50° C., BPR: 100 bar; Inj. Vol.: 3.0 uL; UV 210 nm, Sample prep.: 2 mg/ml EtOH. Retention time: 3.76 min (S)-[(2R)-pyrrolidin-2-yl]-(3,4,5-trifluorophenyl)methanol hydrochloride

Chiral SFC method: Column: Chiralcel OZ-3, 3 um, 4.6 mm×150 mm, Nr. 183; Mobile phases, A: CO₂, 90%-60% in 8.8 min, hold for 0.5 min, B: EtOH+0.2% IPAm, 10-40% in 8.8 min. hold for 0.5 min; Flow: 3.0 mL/min; Temp: 50° C., BPR: 220 bar; Inj. Vol.: 3.0 uL; UV 210 nm, Sample prep.: 2 mg/ml methanol. Retention time: 2.11 min (S)-[(2R)-pyrrolidin-2-yl]-(3,4,5-trifluorophenyl)methanol hydrochloride

1. A process for the preparation of a chiral pyrollidine-2-yl-methanol derivative of formula I 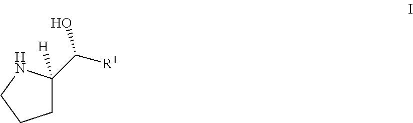
or a salt thereof, wherein
R¹ is aryl or heteroaryl, each optionally substituted with one or more substituents selected from the group consisting of C₁₋₄-alkyl, halo-C₁₋₄-alkyl, C₁₋₄-alkoxy and halogen,
said process comprising
a) transforming a pyrrolidine carboxylic acid derivative of formula II 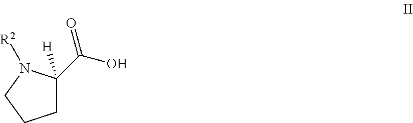
with an N,O-dialkylhydroxylamine of formula V
R⁴ONHR³ V
into the carbamoyl pyrrolidine derivative of formula III 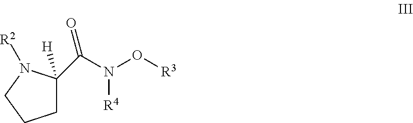
wherein R² is an amino protecting group; and R³ and R⁴ are each independently C₁₋₄-alkyl;
b) reacting the carbamoyl pyrrolidine derivative of formula III with a Grignard reagent of the formula
R¹MgHal
to form the aroyl pyrrolidine derivative of formula IV 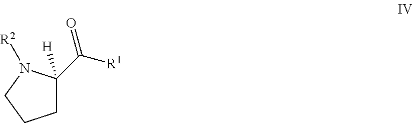
wherein
R¹ is aryl or heteroaryl, each optionally substituted with one or more substituents selected from the group consisting of C₁₋₄-alkyl, halo-C₁₋₄-alkyl, C₁₋₄-alkoxy and halogen;
Hal is halogen; and
R² is an amino protecting group; and
c) removing the amino protecting group R² from the aroyl pyrrolidine derivative of formula IV, and subsequently hydrogenating the aroyl pyrrolidine derivative in the presence of a hydrogenation catalyst to form the chiral pyrollidine-2-yl-methanol derivative of formula I.

2. The process of claim 1, wherein the chiral pyrollidine-2-yl-methanol derivative has the structure of formula Ia. 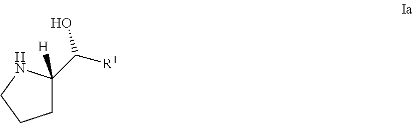
wherein R¹ is aryl or heteroaryl, each optionally substituted with one or more substituents selected from the group consisting of C₁₋₄-alkyl, halo-C₁₋₄-alkyl, C₁₋₄-alkoxy and halogen.

3. The process of claim 1, wherein the chiral pyrollidine-2-yl-methanol derivative the structure of formula Ib. 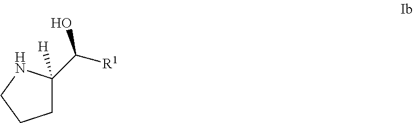
wherein R¹ is aryl or heteroaryl, each optionally substituted with one or more substituents selected from the group consisting of C₁₋₄-alkyl, halo-C₁₋₄-alkyl, C₁₋₄-alkoxy and halogen.

4. The process of claim 1, wherein R¹ is aryl, optionally substituted with one or more substituents selected from the group consisting of C₁₋₄-alkyl, halo-C₁₋₄-alkyl and C₁₋₄-alkoxy.

5. The process of claim 4, wherein R¹ is phenyl.

6. The process of claim 1, wherein the transformation in step a) is performed in the presence of a coupling agent, an amine base and an organic solvent at a reaction temperature between 0° C. and 60° C.

7. The process of claim 6, wherein the coupling agent is selected from the group consisting of n-propylphosphonic acid anhydride (T3P®), N,N′-dicyclohexylcarbodiimide (DCC), N-(3-dimethylaminopropyl)-N′-ethylcarbodiimide-hydrochloride (EDC), N,N,N′,N′-tetramethyl-O-(benzotriazol-1-yl)uronium tetrafluoroborate (TBTU), and 2-(1H-benzotriazol-1-yl)-1,1,3,3-tetramethyluronium hexafluorophosphate (HBTU).

8. The process of claim 7, wherein the coupling agent is combined with an additive selected from the group consisting of 1-hydroxybenztriazole (HOBt), N-hydroxysuccinimide (HOSu), and 1-hydroxy-7-azabenzotriazole (HOAt) and combinations thereof.

9. The process of claim 6, wherein the amine base is a tertiary amine, and the organic solvent is a polar aprotic solvent.

10. The process of claim 1, wherein step b) is performed in an organic solvent at a reaction temperature between −10° C. and 50° C.

11. The process of claim 10, wherein the organic solvent is an ethereal or aromatic hydrocarbon solvent or mixtures thereof.

12. The process of claim 1, wherein the amino protecting group R² is cleavable under acidic conditions.

13. The process of claim 1, wherein R² is tert-butoxycarbonyl (BOC).

14. The process of claim 1, wherein removing the amino protecting group R² from the aroyl pyrrolidine derivative of formula IV is performed with a strong acid.

15. The process of claim 14, wherein the strong acid is hydrochloric acid.

16. The process of claim 1, wherein the hydrogenation in step c) is performed in the presence of a hydrogenation catalyst comprising a platinum group metal selected from the group consisting of ruthenium, osmium, rhodium, iridium, palladium and platinum.

17. The process of claim 1, wherein the platinum group metal is palladium.

18. The process of claim 1, wherein the hydrogenation in step c) is performed in a polar protic solvent at a reaction temperature between 0° C. and 60° C. and a hydrogen pressure between 1 and 10 bar.

19. The process of claim 1, wherein the chiral pyrollidine-2-yl-methanol derivative is obtained in the form of its hydrochloride salt.

20. The process of claim 1, wherein the chiral pyrollidine-2-yl-methanol derivative is selected from the group consisting of [chemical structure]
